(19) United States
(12) Patent Application Publication
(10) Pub. No.: US 2023/0052318 A1
(43) Pub. Date: Feb. 16, 2023

(54) TREATMENT OF PAIN ASSOCIATED WITH CESAREAN SECTION SURGERY WITH SUSTAINED-RELEASE LIPOSOMAL ANESTHETIC COMPOSITIONS

(71) Applicant: Pacira Pharmaceuticals, Inc., Parsippany, NJ (US)

(72) Inventors: Roy Winston, Parsippany, NJ (US); Richard Scranton, Parsippany, NJ (US); Kathy Los, Parsippany, NJ (US); Vladimir Kharitonov, Parsippany, NJ (US)

(21) Appl. No.: 17/789,704

(22) PCT Filed: Jan. 6, 2021
(86) PCT No.: PCT/US2021/012266

§ 371 Date: Jun. 28, 2022

Related U.S. Application Data
(60) Provisional application No. 62/957,694, filed on Jan. 6, 2020.
(60) Provisional application No. 63/064,760, filed on Aug. 12, 2020.
(60) Provisional application No. 63/066,573, filed on Aug. 17, 2020.

(51) Int. Cl. A61K 31/167; A61K 9/127; A61K 31/661; A61P 23/00

(52) U.S. Cl. CPC A61K 31/167; A61K 9/127; A61K 31/661; A61P 23/00

(57) ABSTRACT

In some embodiments provided herein is a method of treating pain associated with cesarean section surgery in a subject, the method comprising administering into the subject a pharmaceutical composition comprising: a) a multivesicular liposome comprising: at least one amphipathic lipid, and at least one neutral lipid; and b) an aqueous phase comprising bupivacaine phosphate, wherein the aqueous phase is encapsulated within the multivesicular liposome.

### CROSS-REFERENCE TO RELATED APPLICATIONS

This application claims the benefit of U.S. Provisional Application Ser. No. 62/957,694, filed Jan. 6, 2020, U.S. Provisional Application Ser. No. 63/064,760, filed Aug. 12, 2020, and U.S. Provisional Application Ser. No. 63/066,573, filed Aug. 17, 2020, each of which is incorporated by reference herein in its entirety.

### BACKGROUND

Cesarean delivery is a common surgical procedure, accounting for 32% of all births in the United States and ˜20% globally. In the United States, ˜1.2 million cesarean deliveries are performed each year, making it the most common surgery after cataract surgery. Inadequately controlled pain following cesarean delivery may interfere with infant bonding, delay recovery, and reduce breastfeeding success. Additionally, women who undergo cesarean delivery are at increased risk for long-term opioid use. Thus, an important goal following cesarean delivery is to improve analgesia while reducing opioid consumption.

Multimodal pain management approaches are recommended by professional societies to improve analgesia, reduce opioid use, and decrease opioid-related adverse events (AEs) following cesarean delivery. It is recommended that protocols include long-acting neuraxial opioids together with scheduled acetaminophen and nonsteroidal anti-inflammatory drugs (NSAIDs). However, most women still request opioids after cesarean delivery for breakthrough pain. Local anesthetic techniques including wound infiltration and truncal blocks, such as transversus abdominis plane (TAP) block, may be of benefit in women not receiving intrathecal morphine. However, benefit appears to be limited when TAP block is used in women receiving intrathecal morphine. This is possibly related to the short duration of analgesia (˜5-8 hours) of standard local anesthetics, such as bupivacaine (BUPI) hydrochloride (HCl).

Thus, there continues to be a need for methods of treating pain associated with cesarean section surgery in a subject.

### SUMMARY

In some embodiments provided herein is a method of treating pain associated with cesarean section surgery in a subject, the method comprising administering into the subject a pharmaceutical composition comprising: a) a multivesicular liposome comprising: at least one amphipathic lipid, and at least one neutral lipid; and b) an aqueous phase comprising: at least one polyhydroxy carboxylic acid and at least one di- or tri-protic mineral acid; and bupivacaine or a salt thereof, such as the hydrochloride salt, wherein the aqueous phase is encapsulated within the multivesicular liposome.

In some embodiments provided herein is a method of treating pain associated with cesarean section surgery in a subject, the method comprising administering into the subject a pharmaceutical composition comprising: a) a multivesicular liposome comprising: at least one amphipathic lipid, and at least one neutral lipid; and b) an aqueous phase comprising: at least one polyhydroxy carboxylic acid and at least one di- or tri-protic mineral acid; and bupivacaine phosphate, wherein the aqueous phase is encapsulated within the multivesicular liposome.

In some embodiments provided herein is a method of treating pain associated with cesarean section surgery in a subject, the method comprising administering into the subject a pharmaceutical composition comprising: a) a multivesicular liposome comprising: at least one amphipathic lipid, and at least one neutral lipid; and b) an aqueous phase comprising bupivacaine phosphate, wherein the aqueous phase is encapsulated within the multivesicular liposome.

In some embodiments provided herein is a method of treating pain associated with cesarean section surgery in a subject, the method comprising administering into the subject a pharmaceutical composition comprising multivesicular liposomes encapsulating bupivacaine phosphate, said multivesicular liposomes comprising:

bupivacaine or a salt thereof;

phosphoric acid;

a lipid component comprising at least one amphipathic lipid and at least one neutral lipid lacking a hydrophilic head group; and,

optionally, a cholesterol and/or a plant sterol wherein said multivesicular liposomes are made by a process comprising:

a) preparing a first aqueous component comprising phosphoric acid;

b) preparing a lipid component comprising at least one organic solvent, at least one amphipathic lipid, and at least one neutral lipid lacking a hydrophilic head group;

c) mixing said first aqueous component and said lipid component to form a water-in-oil emulsion, wherein at least one component comprises bupivacaine or a salt thereof;

d) mixing said water-in-oil emulsion with a second aqueous component to form solvent spherules; and

e) removing the organic solvent from the solvent spherules to form multivesicular liposomes encapsulating bupivacaine phosphate.

In some embodiments provided herein is a method of treating pain associated with cesarean section surgery in a subject, the method comprising administering into the subject a multivesicular liposomal particle pharmaceutical composition made by a process comprising: a) providing a volume of first emulsion by mixing a volume of a first aqueous phase and a volume of a volatile water-immiscible solvent phase, said solvent phase comprising at least one amphipathic lipid and at least one neutral lipid; b) mixing and emulsifying said first emulsion and a volume of a second aqueous phase in a high shear mixer to provide a volume of a second emulsion, said second emulsion comprising a continuous aqueous phase; and c) removing the volatile water-immiscible solvent from the second emulsion to form a volume of multivesicular liposomal particle composition, wherein said solvent removal comprises contacting the second emulsion with an inert gas flow; and wherein said process further comprises primary filtration of the multivesicular liposomal particle composition by cross-flow filtration using a filter having a membrane where the multivesicular liposomal particle composition does not pass through the membrane; wherein all steps are carried out under aseptic conditions, and wherein all solutions are sterile filtered, and wherein the multivesicular liposomal particle composition is immediately suitable for administration into humans; and wherein the primary filtration comprises: a first concentration of the multivesicular liposomal particle composition; and a buffer exchange, resulting in a pH of the multivesicular liposomal particle composition of between about 5 and about 8, and the primary filtration is conducted at a transmembrane pressure of from about 0.1 psi to about 20 psi, such as to about 7 psi.

In some embodiments provided herein is a method of treating pain associated with cesarean section surgery in a subject, the method comprising administering into the subject a multivesicular liposomal particle pharmaceutical composition of pre-determined, uniform size distribution, made by a process comprising: a) providing a first emulsion by mixing a first aqueous phase and a volatile water-immiscible solvent phase, said solvent phase comprising at least one amphipathic lipid and at least one neutral lipid; b) mixing and emulsifying said first emulsion and a second aqueous phase in a mixer to provide a second emulsion, said second emulsion comprising a continuous aqueous phase; c) sparging the volatile water-immiscible solvent from the second emulsion to form an aqueous suspension of multivesicular liposomal particles by bubbling an inert gas through the second emulsion using at least one sparge ring, at least one sparge tube or at least one fit; d) primary filtration of the aqueous suspension of multivesicular liposomal particles by cross-flow filtration using a filter to exchange the second aqueous phase with an aqueous component to provide an initial volume of aqueous media, wherein the filter has a membrane pore size from 0.07 to 0.45 μm; e) secondary filtration by cross-flow filtration to reduce the initial volume to provide a subsequent volume of aqueous media that is 10% to 90% of the initial volume, further wherein the cross-flow filtration is carried out with a process-scale tangential flow filter with a filtration area of 23 square feet or more, wherein all steps are carried out under aseptic conditions, f) the composition is prepared in quantities or batches greater than a liter; wherein the first emulsion is mixed in a first emulsification vessel of at least 10 liters in volume; and g) wherein the uniform size distribution has a number weighted mean particle size of at least 10 microns.

In some embodiments provided herein is a method of treating pain associated with cesarean section surgery in a subject, the method comprising administering into the subject a composition comprising multivesicular liposomes comprising bupivacaine or a salt thereof and having a structure including multiple non-concentric chambers and comprising at least one amphipathic lipid and at least one neutral lipid, wherein said multivesicular liposomes are made by a process comprising removing organic solvent from multivesicular liposomes pre-droplets that comprise a first component core and an aqueous phase shell with an evaporation apparatus, the evaporation apparatus comprising: a solvent removal vessel having a top, a bottom and a circular wall; at least one atomizing nozzle; a carrier gas entrance orifice; a solvent removal gas exit orifice centrally connected to the top; and a product exit orifice connected to the bottom of the vessel,

wherein the process comprises: introducing the pre-droplets to the solvent removal vessel; applying a carrier gas in a tangential direction to the circular wall through the carrier gas entrance orifice; and removing a solvent removal gas through the solvent removal gas exit orifice to provide the large diameter synthetic membrane vesicles.

In some embodiments, the method of treating pain associated with cesarean section surgery in a subject comprises administering an opioid to the subject following completion of the cesarean section surgery.

In some embodiments, the opioid is administered in a total amount less than 50 mg in the first about 72 hours following completion of the cesarean section surgery.

In some embodiments of the method of treating pain associated with cesarean section surgery in a subject, wherein the subject is a first subject, in the first about 72 hours following completion of the cesarean section surgery the opioid is administered to the first subject in a total amount that is lower than the total amount of an opioid that is administered to a second subject in the first about 72 hours following completion of a cesarean section surgery in the second subject, wherein a pharmaceutical composition comprising: a) a multivesicular liposome comprising: at least one amphipathic lipid, and at least one neutral lipid; and b) an aqueous phase comprising: at least one polyhydroxy carboxylic acid and at least one di- or tri-protic mineral acid; and bupivacaine phosphate, wherein the aqueous phase is encapsulated within the multivesicular liposome, is not administered to the second subject.

In some embodiments of the method of treating pain associated with cesarean section surgery in a subject, wherein the subject is a first subject, in the first about 72 hours following completion of the cesarean section surgery the opioid is administered to the first subject in a total amount that is lower than the total amount of an opioid that is administered to a second subject in the first about 72 hours following completion of a cesarean section surgery in the second subject, wherein a pharmaceutical composition comprising: a) a multivesicular liposome comprising: at least one amphipathic lipid, and at least one neutral lipid; and b) an aqueous phase comprising bupivacaine phosphate, wherein the aqueous phase is encapsulated within the multivesicular liposome, is not administered to the second subject.

In some embodiments of the method of treating pain associated with cesarean section surgery in a subject, wherein the subject is a first subject, in the first about 72 hours following completion of the cesarean section surgery the opioid is administered to the first subject in a total amount that is lower than the total amount of an opioid that is administered to a second subject in the first about 72 hours following completion of a cesarean section surgery in the second subject, wherein a pharmaceutical composition comprising multivesicular liposomes encapsulating bupivacaine phosphate, said multivesicular liposomes comprising:

bupivacaine or a salt thereof;

phosphoric acid;

a lipid component comprising at least one amphipathic lipid and at least one neutral lipid lacking a hydrophilic head group; and,

optionally, a cholesterol and/or a plant sterol wherein said multivesicular liposomes are made by a process comprising:

a) preparing a first aqueous component comprising phosphoric acid;

b) preparing a lipid component comprising at least one organic solvent, at least one amphipathic lipid, and at least one neutral lipid lacking a hydrophilic head group;

c) mixing said first aqueous component and said lipid component to form a water-in-oil emulsion, wherein at least one component comprises bupivacaine or a salt thereof;

d) mixing said water-in-oil emulsion with a second aqueous component to form solvent spherules; and

e) removing the organic solvent from the solvent spherules to form multivesicular liposomes encapsulating bupivacaine phosphate,

is not administered to the second subject.

In some embodiments of the method of treating pain associated with cesarean section surgery in a subject, wherein the subject is a first subject, in the first about 72 hours following completion of the cesarean section surgery the opioid is administered to the first subject in a total amount that is lower than the total amount of an opioid that is administered to a second subject in the first about 72 hours following completion of a cesarean section surgery in the second subject,

wherein a multivesicular liposomal particle pharmaceutical composition made by a process comprising: a) providing a volume of first emulsion by mixing a volume of a first aqueous phase and a volume of a volatile water-immiscible solvent phase, said solvent phase comprising at least one amphipathic lipid and at least one neutral lipid; b) mixing and emulsifying said first emulsion and a volume of a second aqueous phase in a high shear mixer to provide a volume of a second emulsion, said second emulsion comprising a continuous aqueous phase; and c) removing the volatile water-immiscible solvent from the second emulsion to form a volume of multivesicular liposomal particle composition, wherein said solvent removal comprises contacting the second emulsion with an inert gas flow; and wherein said process further comprises primary filtration of the multivesicular liposomal particle composition by cross-flow filtration using a filter having a membrane where the multivesicular liposomal particle composition does not pass through the membrane; wherein all steps are carried out under aseptic conditions, and wherein all solutions are sterile filtered, and wherein the multivesicular liposomal particle composition is immediately suitable for administration into humans; and wherein the primary filtration comprises: a first concentration of the multivesicular liposomal particle composition; and a buffer exchange, resulting in a pH of the multivesicular liposomal particle composition of between about 5 and about 8, and the primary filtration is conducted at a transmembrane pressure of from about 0.1 psi to about 20 psi, such as to about 7 psi, is not administered to the second subject.

In some embodiments of the method of treating pain associated with cesarean section surgery in a subject, wherein the subject is a first subject, in the first about 72 hours following completion of the cesarean section surgery the opioid is administered to the first subject in a total amount that is lower than the total amount of an opioid that is administered to a second subject in the first about 72 hours following completion of a cesarean section surgery in the second subject,

wherein a multivesicular liposomal particle pharmaceutical composition of pre-determined, uniform size distribution, made by a process comprising: a) providing a first emulsion by mixing a first aqueous phase and a volatile water-immiscible solvent phase, said solvent phase comprising at least one amphipathic lipid and at least one neutral lipid; b) mixing and emulsifying said first emulsion and a second aqueous phase in a mixer to provide a second emulsion, said second emulsion comprising a continuous aqueous phase; c) sparging the volatile water-immiscible solvent from the second emulsion to form an aqueous suspension of multivesicular liposomal particles by bubbling an inert gas through the second emulsion using at least one sparge ring, at least one sparge tube or at least one fit; d) primary filtration of the aqueous suspension of multivesicular liposomal particles by cross-flow filtration using a filter to exchange the second aqueous phase with an aqueous component to provide an initial volume of aqueous media, wherein the filter has a membrane pore size from 0.07 to 0.45 μm; e) secondary filtration by cross-flow filtration to reduce the initial volume to provide a subsequent volume of aqueous media that is 10% to 90% of the initial volume, further wherein the cross-flow filtration is carried out with a process-scale tangential flow filter with a filtration area of 23 square feet or more, wherein all steps are carried out under aseptic conditions, f) the composition is prepared in quantities or batches greater than a liter; wherein the first emulsion is mixed in a first emulsification vessel of at least 10 liters in volume; and g) wherein the uniform size distribution has a number weighted mean particle size of at least 10 microns,

is not administered to the second subject.

In some embodiments of the method of treating pain associated with cesarean section surgery in a subject, wherein the subject is a first subject, in the first about 72 hours following completion of the cesarean section surgery the opioid is administered to the first subject in a total amount that is lower than the total amount of an opioid that is administered to a second subject in the first about 72 hours following completion of a cesarean section surgery in the second subject, wherein a composition comprising multivesicular liposomes comprising bupivacaine or a salt thereof and having a structure including multiple non-concentric chambers and comprising at least one amphipathic lipid and at least one neutral lipid, wherein said multivesicular liposomes are made by a process comprising removing organic solvent from multivesicular liposomes pre-droplets that comprise a first component core and an aqueous phase shell with an evaporation apparatus, the evaporation apparatus comprising: a solvent removal vessel having a top, a bottom and a circular wall; at least one atomizing nozzle; a carrier gas entrance orifice; a solvent removal gas exit orifice centrally connected to the top; and a product exit orifice connected to the bottom of the vessel,

wherein the process comprises: introducing the pre-droplets to the solvent removal vessel; applying a carrier gas in a tangential direction to the circular wall through the carrier gas entrance orifice; and removing a solvent removal gas through the solvent removal gas exit orifice to provide the large diameter synthetic membrane vesicles,

is not administered to the second subject.

### BRIEF DESCRIPTION OF THE DRAWINGS

FIG. 1 shows the patient disposition in the study disclosed herein. Efficacy was evaluated in a modified intent-to-treat population that included patients who underwent cesarean delivery and met the study criteria for correct transversus abdominis plane placement, local anesthetic dosing, and multimodal postsurgical analgesic regimen. Those that did not meet ≥1 criteria for correct TAP placement (n=13), local anesthetic dosing (n=12), or multimodal postsurgical analgesic regimen (n=39) were excluded from the efficacy analysis. In the figure, BUPI=bupivacaine; HCl=hydrochloride; LB=multivesicular liposomal bupivacaine. Multivesicular liposomal bupivacaine is also referred to as “MVL” herein.

FIG. 2 shows the total opioid consumption through 24, 48, and 72 hours and at 7 and 14 days after cesarean section surgery. In the figure, BUPI=bupivacaine; HCl=hydrochloride; LB=multivesicular liposomal bupivacaine; LSM=least squares mean; MED=morphine equivalent dose.

FIG. 3 shows the total opioid consumption through 72 hours after surgery for cesarean delivery. In the figure, HCl=hydrochloride; LB=multivesicular liposomal bupivacaine, LSM=least squares mean; MED=morphine equivalent dose.

FIG. 4 shows the total opioid consumption through 24 hours, 48 hours, 7 days, and 14 days after surgery for cesarean delivery. In the figure, HCl=hydrochloride; LB=multivesicular liposomal bupivacaine, LSM=least squares mean; MED=morphine equivalent dose.

FIG. 5 shows the percentage of opioid-spared patients at 72 hours after cesarean section surgery. In the figure, HCl=hydrochloride; LB=multivesicular liposomal bupivacaine. Opioid sparing was defined as taking ≤15 mg of oxycodone (or equivalent) with an OBAS score of 0 for questions 2, 3, 4, 5, and 6 through 72 h in a prespecified analysis. OBAS=Overall Benefit of Anesthesia Score.

### DETAILED DESCRIPTION

In some embodiments provided herein is a method of treating pain associated with cesarean section surgery in a subject, the method comprising administering into the subject a pharmaceutical composition comprising: a) a multivesicular liposome comprising: at least one amphipathic lipid, and at least one neutral lipid; and b) an aqueous phase comprising: at least one polyhydroxy carboxylic acid and at least one di- or tri-protic mineral acid; and bupivacaine phosphate, wherein the aqueous phase is encapsulated within the multivesicular liposome.

In some embodiments provided herein is a method of treating pain associated with cesarean section surgery in a subject, the method comprising administering into the subject a pharmaceutical composition comprising: a) a multivesicular liposome comprising: at least one amphipathic lipid, and at least one neutral lipid; and b) an aqueous phase comprising bupivacaine phosphate, wherein the aqueous phase is encapsulated within the multivesicular liposome.

In some embodiments provided herein is a method of treating pain associated with cesarean section surgery in a subject, the method comprising administering into the subject a pharmaceutical composition comprising multivesicular liposomes encapsulating bupivacaine phosphate, said multivesicular liposomes comprising:

bupivacaine or a salt thereof;

phosphoric acid;

a lipid component comprising at least one amphipathic lipid and at least one neutral lipid lacking a hydrophilic head group; and,

optionally, a cholesterol and/or a plant sterol wherein said multivesicular liposomes are made by a process comprising:

a) preparing a first aqueous component comprising phosphoric acid;

b) preparing a lipid component comprising at least one organic solvent, at least one amphipathic lipid, and at least one neutral lipid lacking a hydrophilic head group;

c) mixing said first aqueous component and said lipid component to form a water-in-oil emulsion, wherein at least one component comprises bupivacaine or a salt thereof;

d) mixing said water-in-oil emulsion with a second aqueous component to form solvent spherules; and

e) removing the organic solvent from the solvent spherules to form multivesicular liposomes encapsulating bupivacaine phosphate.

In some embodiments provided herein is a method of treating pain associated with cesarean section surgery in a subject, the method comprising administering into the subject a multivesicular liposomal particle pharmaceutical composition made by a process comprising: a) providing a volume of first emulsion by mixing a volume of a first aqueous phase and a volume of a volatile water-immiscible solvent phase, said solvent phase comprising at least one amphipathic lipid and at least one neutral lipid; b) mixing and emulsifying said first emulsion and a volume of a second aqueous phase in a high shear mixer to provide a volume of a second emulsion, said second emulsion comprising a continuous aqueous phase; and c) removing the volatile water-immiscible solvent from the second emulsion to form a volume of multivesicular liposomal particle composition, wherein said solvent removal comprises contacting the second emulsion with an inert gas flow; and wherein said process further comprises primary filtration of the multivesicular liposomal particle composition by cross-flow filtration using a filter having a membrane where the multivesicular liposomal particle composition does not pass through the membrane; wherein all steps are carried out under aseptic conditions, and wherein all solutions are sterile filtered, and wherein the multivesicular liposomal particle composition is immediately suitable for administration into humans; and wherein the primary filtration comprises: a first concentration of the multivesicular liposomal particle composition; and a buffer exchange, resulting in a pH of the multivesicular liposomal particle composition of between about 5 and about 8, and the primary filtration is conducted at a transmembrane pressure of from about 0.1 psi to about 20 psi, such as to about 7 psi.

In some embodiments provided herein is a method of treating pain associated with cesarean section surgery in a subject, the method comprising administering into the subject a multivesicular liposomal particle pharmaceutical composition of pre-determined, uniform size distribution, made by a process comprising: a) providing a first emulsion by mixing a first aqueous phase and a volatile water-immiscible solvent phase, said solvent phase comprising at least one amphipathic lipid and at least one neutral lipid; b) mixing and emulsifying said first emulsion and a second aqueous phase in a mixer to provide a second emulsion, said second emulsion comprising a continuous aqueous phase; c) sparging the volatile water-immiscible solvent from the second emulsion to form an aqueous suspension of multivesicular liposomal particles by bubbling an inert gas through the second emulsion using at least one sparge ring, at least one sparge tube or at least one fit; d) primary filtration of the aqueous suspension of multivesicular liposomal particles by cross-flow filtration using a filter to exchange the second aqueous phase with an aqueous component to provide an initial volume of aqueous media, wherein the filter has a membrane pore size from 0.07 to 0.45 um; e) secondary filtration by cross-flow filtration to reduce the initial volume to provide a subsequent volume of aqueous media that is 10% to 90% of the initial volume, further wherein the cross-flow filtration is carried out with a process-scale tangential flow filter with a filtration area of 23 square feet or more, wherein all steps are carried out under aseptic conditions, f) the composition is prepared in quantities or batches greater than a liter; wherein the first emulsion is mixed in a first emulsification vessel of at least 10 liters in volume; and g) wherein the uniform size distribution has a number weighted mean particle size of at least 10 microns.

In some embodiments provided herein is a method of treating pain associated with cesarean section surgery in a subject, the method comprising administering into the subject a composition comprising multivesicular liposomes comprising bupivacaine or a salt thereof and having a structure including multiple non-concentric chambers and comprising at least one amphipathic lipid and at least one neutral lipid, wherein said multivesicular liposomes are made by a process comprising removing organic solvent from multivesicular liposomes pre-droplets that comprise a first component core and an aqueous phase shell with an evaporation apparatus, the evaporation apparatus comprising: a solvent removal vessel having a top, a bottom and a circular wall; at least one atomizing nozzle; a carrier gas entrance orifice; a solvent removal gas exit orifice centrally connected to the top; and a product exit orifice connected to the bottom of the vessel,

wherein the process comprises: introducing the pre-droplets to the solvent removal vessel; applying a carrier gas in a tangential direction to the circular wall through the carrier gas entrance orifice; and removing a solvent removal gas through the solvent removal gas exit orifice to provide the large diameter synthetic membrane vesicles.

In some embodiments provided herein is a method of anesthetizing a subject in need of anesthetization prior to cesarean section surgery, the method comprising administering into the subject a pharmaceutical composition comprising: a) a multivesicular liposome comprising: at least one amphipathic lipid, and at least one neutral lipid; and b) an aqueous phase comprising: at least one polyhydroxy carboxylic acid and at least one di- or tri-protic mineral acid; and bupivacaine phosphate, wherein the aqueous phase is encapsulated within the multivesicular liposome.

In some embodiments provided herein is a method of anesthetizing a subject in need of anesthetization prior to cesarean section surgery, the method comprising administering into the subject a pharmaceutical composition comprising: a) a multivesicular liposome comprising: at least one amphipathic lipid, and at least one neutral lipid; and b) an aqueous phase comprising bupivacaine phosphate, wherein the aqueous phase is encapsulated within the multivesicular liposome.

In some embodiments provided herein is a method of anesthetizing a subject in need of anesthetization prior to cesarean section surgery, the method comprising administering into the subject a pharmaceutical composition comprising multivesicular liposomes encapsulating bupivacaine phosphate, said multivesicular liposomes comprising:

bupivacaine or a salt thereof;

phosphoric acid;

a lipid component comprising at least one amphipathic lipid and at least one neutral lipid lacking a hydrophilic head group; and,

optionally, a cholesterol and/or a plant sterol wherein said multivesicular liposomes are made by a process comprising:

a) preparing a first aqueous component comprising phosphoric acid;

b) preparing a lipid component comprising at least one organic solvent, at least one amphipathic lipid, and at least one neutral lipid lacking a hydrophilic head group;

c) mixing said first aqueous component and said lipid component to form a water-in-oil emulsion, wherein at least one component comprises bupivacaine or a salt thereof;

d) mixing said water-in-oil emulsion with a second aqueous component to form solvent spherules; and

e) removing the organic solvent from the solvent spherules to form multivesicular liposomes encapsulating bupivacaine phosphate.

In some embodiments provided herein is a method of anesthetizing a subject in need of anesthetization prior to cesarean section surgery, the method comprising administering into the subject a multivesicular liposomal particle pharmaceutical composition made by a process comprising: a) providing a volume of first emulsion by mixing a volume of a first aqueous phase and a volume of a volatile water-immiscible solvent phase, said solvent phase comprising at least one amphipathic lipid and at least one neutral lipid; b) mixing and emulsifying said first emulsion and a volume of a second aqueous phase in a high shear mixer to provide a volume of a second emulsion, said second emulsion comprising a continuous aqueous phase; and c) removing the volatile water-immiscible solvent from the second emulsion to form a volume of multivesicular liposomal particle composition, wherein said solvent removal comprises contacting the second emulsion with an inert gas flow; and wherein said process further comprises primary filtration of the multivesicular liposomal particle composition by cross-flow filtration using a filter having a membrane where the multivesicular liposomal particle composition does not pass through the membrane; wherein all steps are carried out under aseptic conditions, and wherein all solutions are sterile filtered, and wherein the multivesicular liposomal particle composition is immediately suitable for administration into humans; and wherein the primary filtration comprises: a first concentration of the multivesicular liposomal particle composition; and a buffer exchange, resulting in a pH of the multivesicular liposomal particle composition of between about 5 and about 8, and the primary filtration is conducted at a transmembrane pressure of from about 0.1 psi to about 20 psi, such as to about 7 psi.

In some embodiments provided herein is a method of anesthetizing a subject in need of anesthetization prior to cesarean section surgery, the method comprising administering into the subject a multivesicular liposomal particle pharmaceutical composition of pre-determined, uniform size distribution, made by a process comprising: a) providing a first emulsion by mixing a first aqueous phase and a volatile water-immiscible solvent phase, said solvent phase comprising at least one amphipathic lipid and at least one neutral lipid; b) mixing and emulsifying said first emulsion and a second aqueous phase in a mixer to provide a second emulsion, said second emulsion comprising a continuous aqueous phase; c) sparging the volatile water-immiscible solvent from the second emulsion to form an aqueous suspension of multivesicular liposomal particles by bubbling an inert gas through the second emulsion using at least one sparge ring, at least one sparge tube or at least one fit; d) primary filtration of the aqueous suspension of multivesicular liposomal particles by cross-flow filtration using a filter to exchange the second aqueous phase with an aqueous component to provide an initial volume of aqueous media, wherein the filter has a membrane pore size from 0.07 to 0.45 μm; e) secondary filtration by cross-flow filtration to reduce the initial volume to provide a subsequent volume of aqueous media that is 10% to 90% of the initial volume, further wherein the cross-flow filtration is carried out with a process-scale tangential flow filter with a filtration area of 23 square feet or more, wherein all steps are carried out under aseptic conditions, f) the composition is prepared in quantities or batches greater than a liter; wherein the first emulsion is mixed in a first emulsification vessel of at least 10 liters in volume; and g) wherein the uniform size distribution has a number weighted mean particle size of at least 10 microns.

In some embodiments provided herein is a method of anesthetizing a subject in need of anesthetization prior to cesarean section surgery, the method comprising administering into the subject a composition comprising multivesicular liposomes comprising bupivacaine or a salt thereof and having a structure including multiple non-concentric chambers and comprising at least one amphipathic lipid and at least one neutral lipid, wherein said multivesicular liposomes are made by a process comprising removing organic solvent from multivesicular liposomes pre-droplets that comprise a first component core and an aqueous phase shell with an evaporation apparatus, the evaporation apparatus comprising: a solvent removal vessel having a top, a bottom and a circular wall; at least one atomizing nozzle; a carrier gas entrance orifice; a solvent removal gas exit orifice centrally connected to the top; and a product exit orifice connected to the bottom of the vessel,

wherein the process comprises: introducing the pre-droplets to the solvent removal vessel; applying a carrier gas in a tangential direction to the circular wall through the carrier gas entrance orifice; and removing a solvent removal gas through the solvent removal gas exit orifice to provide the large diameter synthetic membrane vesicles.

In some embodiments provided herein is a method of anesthetizing a subject in need of anesthetization during cesarean section surgery, the method comprising administering into the subject a pharmaceutical composition comprising: a) a multivesicular liposome comprising: at least one amphipathic lipid, and at least one neutral lipid; and b) an aqueous phase comprising: at least one polyhydroxy carboxylic acid and at least one di- or tri-protic mineral acid; and bupivacaine phosphate, wherein the aqueous phase is encapsulated within the multivesicular liposome.

In some embodiments provided herein is a method of anesthetizing a subject in need of anesthetization during cesarean section surgery, the method comprising administering into the subject a pharmaceutical composition comprising: a) a multivesicular liposome comprising: at least one amphipathic lipid, and at least one neutral lipid; and b) an aqueous phase comprising bupivacaine phosphate, wherein the aqueous phase is encapsulated within the multivesicular liposome.

In some embodiments provided herein is a method of anesthetizing a subject in need of anesthetization during cesarean section surgery, the method comprising administering into the subject a pharmaceutical composition comprising multivesicular liposomes encapsulating bupivacaine phosphate, said multivesicular liposomes comprising:

bupivacaine or a salt thereof;

phosphoric acid;

a lipid component comprising at least one amphipathic lipid and at least one neutral lipid lacking a hydrophilic head group; and,

optionally, a cholesterol and/or a plant sterol wherein said multivesicular liposomes are made by a process comprising:

a) preparing a first aqueous component comprising phosphoric acid;

b) preparing a lipid component comprising at least one organic solvent, at least one amphipathic lipid, and at least one neutral lipid lacking a hydrophilic head group;

c) mixing said first aqueous component and said lipid component to form a water-in-oil emulsion, wherein at least one component comprises bupivacaine or a salt thereof;

d) mixing said water-in-oil emulsion with a second aqueous component to form solvent spherules; and

e) removing the organic solvent from the solvent spherules to form multivesicular liposomes encapsulating bupivacaine phosphate.

In some embodiments provided herein is a method of anesthetizing a subject in need of anesthetization during cesarean section surgery, the method comprising administering into the subject a multivesicular liposomal particle pharmaceutical composition made by a process comprising: a) providing a volume of first emulsion by mixing a volume of a first aqueous phase and a volume of a volatile water-immiscible solvent phase, said solvent phase comprising at least one amphipathic lipid and at least one neutral lipid; b) mixing and emulsifying said first emulsion and a volume of a second aqueous phase in a high shear mixer to provide a volume of a second emulsion, said second emulsion comprising a continuous aqueous phase; and c) removing the volatile water-immiscible solvent from the second emulsion to form a volume of multivesicular liposomal particle composition, wherein said solvent removal comprises contacting the second emulsion with an inert gas flow; and wherein said process further comprises primary filtration of the multivesicular liposomal particle composition by cross-flow filtration using a filter having a membrane where the multivesicular liposomal particle composition does not pass through the membrane; wherein all steps are carried out under aseptic conditions, and wherein all solutions are sterile filtered, and wherein the multivesicular liposomal particle composition is immediately suitable for administration into humans; and wherein the primary filtration comprises: a first concentration of the multivesicular liposomal particle composition; and a buffer exchange, resulting in a pH of the multivesicular liposomal particle composition of between about 5 and about 8, and the primary filtration is conducted at a transmembrane pressure of from about 0.1 psi to about 20 psi, such as to about 7 psi.

In some embodiments provided herein is a method of anesthetizing a subject in need of anesthetization during cesarean section surgery, the method comprising administering into the subject a multivesicular liposomal particle pharmaceutical composition of pre-determined, uniform size distribution, made by a process comprising: a) providing a first emulsion by mixing a first aqueous phase and a volatile water-immiscible solvent phase, said solvent phase comprising at least one amphipathic lipid and at least one neutral lipid; b) mixing and emulsifying said first emulsion and a second aqueous phase in a mixer to provide a second emulsion, said second emulsion comprising a continuous aqueous phase; c) sparging the volatile water-immiscible solvent from the second emulsion to form an aqueous suspension of multivesicular liposomal particles by bubbling an inert gas through the second emulsion using at least one sparge ring, at least one sparge tube or at least one fit; d) primary filtration of the aqueous suspension of multivesicular liposomal particles by cross-flow filtration using a filter to exchange the second aqueous phase with an aqueous component to provide an initial volume of aqueous media, wherein the filter has a membrane pore size from 0.07 to 0.45 μm; e) secondary filtration by cross-flow filtration to reduce the initial volume to provide a subsequent volume of aqueous media that is 10% to 90% of the initial volume, further wherein the cross-flow filtration is carried out with a process-scale tangential flow filter with a filtration area of 23 square feet or more, wherein all steps are carried out under aseptic conditions, f) the composition is prepared in quantities or batches greater than a liter; wherein the first emulsion is mixed in a first emulsification vessel of at least 10 liters in volume; and g) wherein the uniform size distribution has a number weighted mean particle size of at least 10 microns.

In some embodiments provided herein is a method of anesthetizing a subject in need of anesthetization during to cesarean section surgery, the method comprising administering into the subject a composition comprising multivesicular liposomes comprising bupivacaine or a salt thereof and having a structure including multiple non-concentric chambers and comprising at least one amphipathic lipid and at least one neutral lipid, wherein said multivesicular liposomes are made by a process comprising removing organic solvent from multivesicular liposomes pre-droplets that comprise a first component core and an aqueous phase shell with an evaporation apparatus, the evaporation apparatus comprising: a solvent removal vessel having a top, a bottom and a circular wall; at least one atomizing nozzle; a carrier gas entrance orifice; a solvent removal gas exit orifice centrally connected to the top; and a product exit orifice connected to the bottom of the vessel,

wherein the process comprises: introducing the pre-droplets to the solvent removal vessel; applying a carrier gas in a tangential direction to the circular wall through the carrier gas entrance orifice; and removing a solvent removal gas through the solvent removal gas exit orifice to provide the large diameter synthetic membrane vesicles.

In some embodiments provided herein is a method of anesthetizing a subject in need of anesthetization following cesarean section surgery, the method comprising administering into the subject a pharmaceutical composition comprising: a) a multivesicular liposome comprising: at least one amphipathic lipid, and at least one neutral lipid; and b) an aqueous phase comprising: at least one polyhydroxy carboxylic acid and at least one di- or tri-protic mineral acid; and bupivacaine phosphate, wherein the aqueous phase is encapsulated within the multivesicular liposome.

In some embodiments provided herein is a method of anesthetizing a subject in need of anesthetization following cesarean section surgery, the method comprising administering into the subject a pharmaceutical composition comprising: a) a multivesicular liposome comprising: at least one amphipathic lipid, and at least one neutral lipid; and b) an aqueous phase comprising bupivacaine phosphate, wherein the aqueous phase is encapsulated within the multivesicular liposome.

In some embodiments provided herein is a method of anesthetizing a subject in need of anesthetization following cesarean section surgery, the method comprising administering into the subject a pharmaceutical composition comprising multivesicular liposomes encapsulating bupivacaine phosphate, said multivesicular liposomes comprising:

bupivacaine or a salt thereof;

phosphoric acid;

a lipid component comprising at least one amphipathic lipid and at least one neutral lipid lacking a hydrophilic head group; and,

optionally, a cholesterol and/or a plant sterol wherein said multivesicular liposomes are made by a process comprising:

a) preparing a first aqueous component comprising phosphoric acid;

b) preparing a lipid component comprising at least one organic solvent, at least one amphipathic lipid, and at least one neutral lipid lacking a hydrophilic head group;

c) mixing said first aqueous component and said lipid component to form a water-in-oil emulsion, wherein at least one component comprises bupivacaine or a salt thereof;

d) mixing said water-in-oil emulsion with a second aqueous component to form solvent spherules; and

e) removing the organic solvent from the solvent spherules to form multivesicular liposomes encapsulating bupivacaine phosphate.

In some embodiments provided herein is a method of anesthetizing a subject in need of anesthetization following cesarean section surgery, the method comprising administering into the subject a multivesicular liposomal particle pharmaceutical composition made by a process comprising: a) providing a volume of first emulsion by mixing a volume of a first aqueous phase and a volume of a volatile water-immiscible solvent phase, said solvent phase comprising at least one amphipathic lipid and at least one neutral lipid; b) mixing and emulsifying said first emulsion and a volume of a second aqueous phase in a high shear mixer to provide a volume of a second emulsion, said second emulsion comprising a continuous aqueous phase; and c) removing the volatile water-immiscible solvent from the second emulsion to form a volume of multivesicular liposomal particle composition, wherein said solvent removal comprises contacting the second emulsion with an inert gas flow; and wherein said process further comprises primary filtration of the multivesicular liposomal particle composition by cross-flow filtration using a filter having a membrane where the multivesicular liposomal particle composition does not pass through the membrane; wherein all steps are carried out under aseptic conditions, and wherein all solutions are sterile filtered, and wherein the multivesicular liposomal particle composition is immediately suitable for administration into humans; and wherein the primary filtration comprises: a first concentration of the multivesicular liposomal particle composition; and a buffer exchange, resulting in a pH of the multivesicular liposomal particle composition of between about 5 and about 8, and the primary filtration is conducted at a transmembrane pressure of from about 0.1 psi to about 20 psi, such as to about 7 psi.

In some embodiments provided herein is a method of anesthetizing a subject in need of anesthetization following cesarean section surgery, the method comprising administering into the subject a multivesicular liposomal particle pharmaceutical composition of pre-determined, uniform size distribution, made by a process comprising: a) providing a first emulsion by mixing a first aqueous phase and a volatile water-immiscible solvent phase, said solvent phase comprising at least one amphipathic lipid and at least one neutral lipid; b) mixing and emulsifying said first emulsion and a second aqueous phase in a mixer to provide a second emulsion, said second emulsion comprising a continuous aqueous phase; c) sparging the volatile water-immiscible solvent from the second emulsion to form an aqueous suspension of multivesicular liposomal particles by bubbling an inert gas through the second emulsion using at least one sparge ring, at least one sparge tube or at least one fit; d) primary filtration of the aqueous suspension of multivesicular liposomal particles by cross-flow filtration using a filter to exchange the second aqueous phase with an aqueous component to provide an initial volume of aqueous media, wherein the filter has a membrane pore size from 0.07 to 0.45 μm; e) secondary filtration by cross-flow filtration to reduce the initial volume to provide a subsequent volume of aqueous media that is 10% to 90% of the initial volume, further wherein the cross-flow filtration is carried out with a process-scale tangential flow filter with a filtration area of 23 square feet or more, wherein all steps are carried out under aseptic conditions, f) the composition is prepared in quantities or batches greater than a liter; wherein the first emulsion is mixed in a first emulsification vessel of at least 10 liters in volume; and g) wherein the uniform size distribution has a number weighted mean particle size of at least 10 microns.

In some embodiments provided herein is a method of anesthetizing a subject in need of anesthetization following cesarean section surgery, the method comprising administering into the subject a composition comprising multivesicular liposomes comprising bupivacaine or a salt thereof and having a structure including multiple non-concentric chambers and comprising at least one amphipathic lipid and at least one neutral lipid, wherein said multivesicular liposomes are made by a process comprising removing organic solvent from multivesicular liposomes pre-droplets that comprise a first component core and an aqueous phase shell with an evaporation apparatus, the evaporation apparatus comprising: a solvent removal vessel having a top, a bottom and a circular wall; at least one atomizing nozzle; a carrier gas entrance orifice; a solvent removal gas exit orifice centrally connected to the top; and a product exit orifice connected to the bottom of the vessel,

wherein the process comprises: introducing the pre-droplets to the solvent removal vessel; applying a carrier gas in a tangential direction to the circular wall through the carrier gas entrance orifice; and removing a solvent removal gas through the solvent removal gas exit orifice to provide the large diameter synthetic membrane vesicles.

In some embodiments provided herein is a method of reducing pain associated with cesarean section surgery in a subject, the method comprising administering into the subject a pharmaceutical composition comprising: a) a multivesicular liposome comprising: at least one amphipathic lipid, and at least one neutral lipid; and b) an aqueous phase comprising: at least one polyhydroxy carboxylic acid and at least one di- or tri-protic mineral acid; and bupivacaine phosphate, wherein the aqueous phase is encapsulated within the multivesicular liposome.

In some embodiments provided herein is a method of reducing pain associated with cesarean section surgery in a subject, the method comprising administering into the subject a pharmaceutical composition comprising: a) a multivesicular liposome comprising: at least one amphipathic lipid, and at least one neutral lipid; and b) an aqueous phase comprising bupivacaine phosphate, wherein the aqueous phase is encapsulated within the multivesicular liposome.

In some embodiments provided herein is a method of reducing pain associated with cesarean section surgery in a subject, the method comprising administering into the subject a pharmaceutical composition comprising multivesicular liposomes encapsulating bupivacaine phosphate, said multivesicular liposomes comprising:

bupivacaine or a salt thereof;

phosphoric acid;

a lipid component comprising at least one amphipathic lipid and at least one neutral lipid lacking a hydrophilic head group; and,

optionally, a cholesterol and/or a plant sterol wherein said multivesicular liposomes are made by a process comprising:

a) preparing a first aqueous component comprising phosphoric acid;

b) preparing a lipid component comprising at least one organic solvent, at least one amphipathic lipid, and at least one neutral lipid lacking a hydrophilic head group;

c) mixing said first aqueous component and said lipid component to form a water-in-oil emulsion, wherein at least one component comprises bupivacaine or a salt thereof;

d) mixing said water-in-oil emulsion with a second aqueous component to form solvent spherules; and

e) removing the organic solvent from the solvent spherules to form multivesicular liposomes encapsulating bupivacaine phosphate.

In some embodiments provided herein is a method of reducing pain associated with cesarean section surgery in a subject, the method comprising administering into the subject a multivesicular liposomal particle pharmaceutical composition made by a process comprising: a) providing a volume of first emulsion by mixing a volume of a first aqueous phase and a volume of a volatile water-immiscible solvent phase, said solvent phase comprising at least one amphipathic lipid and at least one neutral lipid; b) mixing and emulsifying said first emulsion and a volume of a second aqueous phase in a high shear mixer to provide a volume of a second emulsion, said second emulsion comprising a continuous aqueous phase; and c) removing the volatile water-immiscible solvent from the second emulsion to form a volume of multivesicular liposomal particle composition, wherein said solvent removal comprises contacting the second emulsion with an inert gas flow; and wherein said process further comprises primary filtration of the multivesicular liposomal particle composition by cross-flow filtration using a filter having a membrane where the multivesicular liposomal particle composition does not pass through the membrane; wherein all steps are carried out under aseptic conditions, and wherein all solutions are sterile filtered, and wherein the multivesicular liposomal particle composition is immediately suitable for administration into humans; and wherein the primary filtration comprises: a first concentration of the multivesicular liposomal particle composition; and a buffer exchange, resulting in a pH of the multivesicular liposomal particle composition of between about 5 and about 8, and the primary filtration is conducted at a transmembrane pressure of from about 0.1 psi to about 20 psi, such as to about 7 psi.

In some embodiments provided herein is a method of reducing pain associated with cesarean section surgery in a subject, the method comprising administering into the subject a multivesicular liposomal particle pharmaceutical composition of pre-determined, uniform size distribution, made by a process comprising: a) providing a first emulsion by mixing a first aqueous phase and a volatile water-immiscible solvent phase, said solvent phase comprising at least one amphipathic lipid and at least one neutral lipid; b) mixing and emulsifying said first emulsion and a second aqueous phase in a mixer to provide a second emulsion, said second emulsion comprising a continuous aqueous phase; c) sparging the volatile water-immiscible solvent from the second emulsion to form an aqueous suspension of multivesicular liposomal particles by bubbling an inert gas through the second emulsion using at least one sparge ring, at least one sparge tube or at least one fit; d) primary filtration of the aqueous suspension of multivesicular liposomal particles by cross-flow filtration using a filter to exchange the second aqueous phase with an aqueous component to provide an initial volume of aqueous media, wherein the filter has a membrane pore size from 0.07 to 0.45 μm; e) secondary filtration by cross-flow filtration to reduce the initial volume to provide a subsequent volume of aqueous media that is 10% to 90% of the initial volume, further wherein the cross-flow filtration is carried out with a process-scale tangential flow filter with a filtration area of 23 square feet or more, wherein all steps are carried out under aseptic conditions, f) the composition is prepared in quantities or batches greater than a liter; wherein the first emulsion is mixed in a first emulsification vessel of at least 10 liters in volume; and g) wherein the uniform size distribution has a number weighted mean particle size of at least 10 microns.

In some embodiments provided herein is a method of reducing pain associated with cesarean section surgery in a subject, the method comprising administering into the subject a composition comprising multivesicular liposomes comprising bupivacaine or a salt thereof and having a structure including multiple non-concentric chambers and comprising at least one amphipathic lipid and at least one neutral lipid, wherein said multivesicular liposomes are made by a process comprising removing organic solvent from multivesicular liposomes pre-droplets that comprise a first component core and an aqueous phase shell with an evaporation apparatus, the evaporation apparatus comprising: a solvent removal vessel having a top, a bottom and a circular wall; at least one atomizing nozzle; a carrier gas entrance orifice; a solvent removal gas exit orifice centrally connected to the top; and a product exit orifice connected to the bottom of the vessel, wherein the process comprises: introducing the pre-droplets to the solvent removal vessel; applying a carrier gas in a tangential direction to the circular wall through the carrier gas entrance orifice; and removing a solvent removal gas through the solvent removal gas exit orifice to provide the large diameter synthetic membrane vesicles.

In some embodiments provided herein is a method of reducing an amount of opioid administered to a subject following cesarean section surgery, the method comprising administering into the subject a pharmaceutical composition comprising: a) a multivesicular liposome comprising: at least one amphipathic lipid, and at least one neutral lipid; and b) an aqueous phase comprising: at least one polyhydroxy carboxylic acid and at least one di- or tri-protic mineral acid; and bupivacaine phosphate, wherein the aqueous phase is encapsulated within the multivesicular liposome.

In some embodiments provided herein is a method of reducing an amount of opioid administered to a subject following cesarean section surgery, the method comprising administering into the subject a pharmaceutical composition comprising: a) a multivesicular liposome comprising: at least one amphipathic lipid, and at least one neutral lipid; and b) an aqueous phase comprising bupivacaine phosphate, wherein the aqueous phase is encapsulated within the multivesicular liposome.

In some embodiments provided herein is a method of reducing a duration of time during which an opioid is administered to a subject following cesarean section surgery, the method comprising administering into the subject a pharmaceutical composition comprising multivesicular liposomes encapsulating bupivacaine phosphate, said multivesicular liposomes comprising:

bupivacaine or a salt thereof;

phosphoric acid;

a lipid component comprising at least one amphipathic lipid and at least one neutral lipid lacking a hydrophilic head group; and,

optionally, a cholesterol and/or a plant sterol wherein said multivesicular liposomes are made by a process comprising:

a) preparing a first aqueous component comprising phosphoric acid;

b) preparing a lipid component comprising at least one organic solvent, at least one amphipathic lipid, and at least one neutral lipid lacking a hydrophilic head group;

c) mixing said first aqueous component and said lipid component to form a water-in-oil emulsion, wherein at least one component comprises bupivacaine or a salt thereof;

d) mixing said water-in-oil emulsion with a second aqueous component to form solvent spherules; and

e) removing the organic solvent from the solvent spherules to form multivesicular liposomes encapsulating bupivacaine phosphate.

In some embodiments provided herein is a method of reducing an amount of opioid administered to a subject following cesarean section surgery, the method comprising administering into the subject a multivesicular liposomal particle pharmaceutical composition made by a process comprising: a) providing a volume of first emulsion by mixing a volume of a first aqueous phase and a volume of a volatile water-immiscible solvent phase, said solvent phase comprising at least one amphipathic lipid and at least one neutral lipid; b) mixing and emulsifying said first emulsion and a volume of a second aqueous phase in a high shear mixer to provide a volume of a second emulsion, said second emulsion comprising a continuous aqueous phase; and c) removing the volatile water-immiscible solvent from the second emulsion to form a volume of multivesicular liposomal particle composition, wherein said solvent removal comprises contacting the second emulsion with an inert gas flow; and wherein said process further comprises primary filtration of the multivesicular liposomal particle composition by cross-flow filtration using a filter having a membrane where the multivesicular liposomal particle composition does not pass through the membrane; wherein all steps are carried out under aseptic conditions, and wherein all solutions are sterile filtered, and wherein the multivesicular liposomal particle composition is immediately suitable for administration into humans; and wherein the primary filtration comprises: a first concentration of the multivesicular liposomal particle composition; and a buffer exchange, resulting in a pH of the multivesicular liposomal particle composition of between about 5 and about 8, and the primary filtration is conducted at a transmembrane pressure of from about 0.1 psi to about 20 psi, such as to about 7 psi.

In some embodiments provided herein is a method of reducing an amount of opioid administered to a subject following cesarean section surgery, the method comprising administering into the subject a multivesicular liposomal particle pharmaceutical composition of pre-determined, uniform size distribution, made by a process comprising: a) providing a first emulsion by mixing a first aqueous phase and a volatile water-immiscible solvent phase, said solvent phase comprising at least one amphipathic lipid and at least one neutral lipid; b) mixing and emulsifying said first emulsion and a second aqueous phase in a mixer to provide a second emulsion, said second emulsion comprising a continuous aqueous phase; c) sparging the volatile water-immiscible solvent from the second emulsion to form an aqueous suspension of multivesicular liposomal particles by bubbling an inert gas through the second emulsion using at least one sparge ring, at least one sparge tube or at least one fit; d) primary filtration of the aqueous suspension of multivesicular liposomal particles by cross-flow filtration using a filter to exchange the second aqueous phase with an aqueous component to provide an initial volume of aqueous media, wherein the filter has a membrane pore size from 0.07 to 0.45 μm; e) secondary filtration by cross-flow filtration to reduce the initial volume to provide a subsequent volume of aqueous media that is 10% to 90% of the initial volume, further wherein the cross-flow filtration is carried out with a process-scale tangential flow filter with a filtration area of 23 square feet or more, wherein all steps are carried out under aseptic conditions, f) the composition is prepared in quantities or batches greater than a liter; wherein the first emulsion is mixed in a first emulsification vessel of at least 10 liters in volume; and g) wherein the uniform size distribution has a number weighted mean particle size of at least 10 microns.

In some embodiments provided herein is a method of reducing an amount of opioid administered to a subject following cesarean section surgery, the method comprising administering into the subject a composition comprising multivesicular liposomes comprising bupivacaine or a salt thereof and having a structure including multiple non-concentric chambers and comprising at least one amphipathic lipid and at least one neutral lipid, wherein said multivesicular liposomes are made by a process comprising removing organic solvent from multivesicular liposomes pre-droplets that comprise a first component core and an aqueous phase shell with an evaporation apparatus, the evaporation apparatus comprising: a solvent removal vessel having a top, a bottom and a circular wall; at least one atomizing nozzle; a carrier gas entrance orifice; a solvent removal gas exit orifice centrally connected to the top; and a product exit orifice connected to the bottom of the vessel,

wherein the process comprises: introducing the pre-droplets to the solvent removal vessel; applying a carrier gas in a tangential direction to the circular wall through the carrier gas entrance orifice; and removing a solvent removal gas through the solvent removal gas exit orifice to provide the large diameter synthetic membrane vesicles.

In some embodiments provided herein is a method of reducing a duration of time during which an opioid is administered to a subject following cesarean section surgery, the method comprising administering into the subject a pharmaceutical composition comprising: a) a multivesicular liposome comprising: at least one amphipathic lipid, and at least one neutral lipid; and b) an aqueous phase comprising: at least one polyhydroxy carboxylic acid and at least one di- or tri-protic mineral acid; and bupivacaine phosphate, wherein the aqueous phase is encapsulated within the multivesicular liposome.

In some embodiments provided herein is a method of reducing a duration of time during which an opioid is administered to a subject following cesarean section surgery, the method comprising administering into the subject a pharmaceutical composition comprising: a) a multivesicular liposome comprising: at least one amphipathic lipid, and at least one neutral lipid; and b) an aqueous phase comprising bupivacaine phosphate, wherein the aqueous phase is encapsulated within the multivesicular liposome.

In some embodiments provided herein is a method of reducing a duration of time during which an opioid is administered to a subject following cesarean section surgery, the method comprising administering into the subject a pharmaceutical composition comprising multivesicular liposomes encapsulating bupivacaine phosphate, said multivesicular liposomes comprising:

bupivacaine or a salt thereof;

phosphoric acid;

a lipid component comprising at least one amphipathic lipid and at least one neutral lipid lacking a hydrophilic head group; and,

optionally, a cholesterol and/or a plant sterol wherein said multivesicular liposomes are made by a process comprising:

a) preparing a first aqueous component comprising phosphoric acid;

b) preparing a lipid component comprising at least one organic solvent, at least one amphipathic lipid, and at least one neutral lipid lacking a hydrophilic head group;

c) mixing said first aqueous component and said lipid component to form a water-in-oil emulsion, wherein at least one component comprises bupivacaine or a salt thereof;

d) mixing said water-in-oil emulsion with a second aqueous component to form solvent spherules; and

e) removing the organic solvent from the solvent spherules to form multivesicular liposomes encapsulating bupivacaine phosphate.

In some embodiments provided herein is a method of reducing a duration of time during which an opioid is administered to a subject following cesarean section surgery, the method comprising administering into the subject a multivesicular liposomal particle pharmaceutical composition made by a process comprising: a) providing a volume of first emulsion by mixing a volume of a first aqueous phase and a volume of a volatile water-immiscible solvent phase, said solvent phase comprising at least one amphipathic lipid and at least one neutral lipid; b) mixing and emulsifying said first emulsion and a volume of a second aqueous phase in a high shear mixer to provide a volume of a second emulsion, said second emulsion comprising a continuous aqueous phase; and c) removing the volatile water-immiscible solvent from the second emulsion to form a volume of multivesicular liposomal particle composition, wherein said solvent removal comprises contacting the second emulsion with an inert gas flow; and wherein said process further comprises primary filtration of the multivesicular liposomal particle composition by cross-flow filtration using a filter having a membrane where the multivesicular liposomal particle composition does not pass through the membrane; wherein all steps are carried out under aseptic conditions, and wherein all solutions are sterile filtered, and wherein the multivesicular liposomal particle composition is immediately suitable for administration into humans; and wherein the primary filtration comprises: a first concentration of the multivesicular liposomal particle composition; and a buffer exchange, resulting in a pH of the multivesicular liposomal particle composition of between about 5 and about 8, and the primary filtration is conducted at a transmembrane pressure of from about 0.1 psi to about 20 psi, such as to about 7 psi.

In some embodiments provided herein is a method of reducing a duration of time during which an opioid is administered to a subject following cesarean section surgery, the method comprising administering into the subject a multivesicular liposomal particle pharmaceutical composition of pre-determined, uniform size distribution, made by a process comprising: a) providing a first emulsion by mixing a first aqueous phase and a volatile water-immiscible solvent phase, said solvent phase comprising at least one amphipathic lipid and at least one neutral lipid; b) mixing and emulsifying said first emulsion and a second aqueous phase in a mixer to provide a second emulsion, said second emulsion comprising a continuous aqueous phase; c) sparging the volatile water-immiscible solvent from the second emulsion to form an aqueous suspension of multivesicular liposomal particles by bubbling an inert gas through the second emulsion using at least one sparge ring, at least one sparge tube or at least one fit; d) primary filtration of the aqueous suspension of multivesicular liposomal particles by cross-flow filtration using a filter to exchange the second aqueous phase with an aqueous component to provide an initial volume of aqueous media, wherein the filter has a membrane pore size from 0.07 to 0.45 μm; e) secondary filtration by cross-flow filtration to reduce the initial volume to provide a subsequent volume of aqueous media that is 10% to 90% of the initial volume, further wherein the cross-flow filtration is carried out with a process-scale tangential flow filter with a filtration area of 23 square feet or more, wherein all steps are carried out under aseptic conditions, f) the composition is prepared in quantities or batches greater than a liter; wherein the first emulsion is mixed in a first emulsification vessel of at least 10 liters in volume; and g) wherein the uniform size distribution has a number weighted mean particle size of at least 10 microns.

In some embodiments provided herein is a method of reducing a duration of time during which an opioid is administered to a subject following cesarean section surgery, the method comprising administering into the subject a composition comprising multivesicular liposomes comprising bupivacaine or a salt thereof and having a structure including multiple non-concentric chambers and comprising at least one amphipathic lipid and at least one neutral lipid, wherein said multivesicular liposomes are made by a process comprising removing organic solvent from multivesicular liposomes pre-droplets that comprise a first component core and an aqueous phase shell with an evaporation apparatus, the evaporation apparatus comprising: a solvent removal vessel having a top, a bottom and a circular wall; at least one atomizing nozzle; a carrier gas entrance orifice; a solvent removal gas exit orifice centrally connected to the top; and a product exit orifice connected to the bottom of the vessel,

wherein the process comprises: introducing the pre-droplets to the solvent removal vessel; applying a carrier gas in a tangential direction to the circular wall through the carrier gas entrance orifice; and removing a solvent removal gas through the solvent removal gas exit orifice to provide the large diameter synthetic membrane vesicles.

In some embodiments provided herein is a method of reducing an amount of a non-opioid analgesic administered to a subject following cesarean section surgery, the method comprising administering into the subject a pharmaceutical composition comprising: a) a multivesicular liposome comprising: at least one amphipathic lipid, and at least one neutral lipid; and b) an aqueous phase comprising: at least one polyhydroxy carboxylic acid and at least one di- or tri-protic mineral acid; and bupivacaine phosphate, wherein the aqueous phase is encapsulated within the multivesicular liposome.

In some embodiments provided herein is a method of reducing an amount of a non-opioid analgesic administered to a subject following cesarean section surgery, the method comprising administering into the subject a pharmaceutical composition comprising: a) a multivesicular liposome comprising: at least one amphipathic lipid, and at least one neutral lipid; and b) an aqueous phase comprising bupivacaine phosphate, wherein the aqueous phase is encapsulated within the multivesicular liposome.

In some embodiments provided herein is a method of reducing an amount of a non-opioid analgesic administered to a subject following cesarean section surgery, the method comprising administering into the subject a pharmaceutical composition comprising multivesicular liposomes encapsulating bupivacaine phosphate, said multivesicular liposomes comprising:

bupivacaine or a salt thereof;

phosphoric acid;

a lipid component comprising at least one amphipathic lipid and at least one neutral lipid lacking a hydrophilic head group; and,

optionally, a cholesterol and/or a plant sterol wherein said multivesicular liposomes are made by a process comprising:

a) preparing a first aqueous component comprising phosphoric acid;

b) preparing a lipid component comprising at least one organic solvent, at least one amphipathic lipid, and at least one neutral lipid lacking a hydrophilic head group;

c) mixing said first aqueous component and said lipid component to form a water-in-oil emulsion, wherein at least one component comprises bupivacaine or a salt thereof;

d) mixing said water-in-oil emulsion with a second aqueous component to form solvent spherules; and

e) removing the organic solvent from the solvent spherules to form multivesicular liposomes encapsulating bupivacaine phosphate.

In some embodiments provided herein is a method of reducing an amount of a non-opioid analgesic administered to a subject following cesarean section surgery, the method comprising administering into the subject a multivesicular liposomal particle pharmaceutical composition made by a process comprising: a) providing a volume of first emulsion by mixing a volume of a first aqueous phase and a volume of a volatile water-immiscible solvent phase, said solvent phase comprising at least one amphipathic lipid and at least one neutral lipid; b) mixing and emulsifying said first emulsion and a volume of a second aqueous phase in a high shear mixer to provide a volume of a second emulsion, said second emulsion comprising a continuous aqueous phase; and c) removing the volatile water-immiscible solvent from the second emulsion to form a volume of multivesicular liposomal particle composition, wherein said solvent removal comprises contacting the second emulsion with an inert gas flow; and wherein said process further comprises primary filtration of the multivesicular liposomal particle composition by cross-flow filtration using a filter having a membrane where the multivesicular liposomal particle composition does not pass through the membrane; wherein all steps are carried out under aseptic conditions, and wherein all solutions are sterile filtered, and wherein the multivesicular liposomal particle composition is immediately suitable for administration into humans; and wherein the primary filtration comprises: a first concentration of the multivesicular liposomal particle composition; and a buffer exchange, resulting in a pH of the multivesicular liposomal particle composition of between about 5 and about 8, and the primary filtration is conducted at a transmembrane pressure of from about 0.1 psi to about 20 psi, such as to about 7 psi.

In some embodiments provided herein is a method of reducing an amount of a non-opioid analgesic administered to a subject following cesarean section surgery, the method comprising administering into the subject a multivesicular liposomal particle pharmaceutical composition of pre-determined, uniform size distribution, made by a process comprising: a) providing a first emulsion by mixing a first aqueous phase and a volatile water-immiscible solvent phase, said solvent phase comprising at least one amphipathic lipid and at least one neutral lipid; b) mixing and emulsifying said first emulsion and a second aqueous phase in a mixer to provide a second emulsion, said second emulsion comprising a continuous aqueous phase; c) sparging the volatile water-immiscible solvent from the second emulsion to form an aqueous suspension of multivesicular liposomal particles by bubbling an inert gas through the second emulsion using at least one sparge ring, at least one sparge tube or at least one fit; d) primary filtration of the aqueous suspension of multivesicular liposomal particles by cross-flow filtration using a filter to exchange the second aqueous phase with an aqueous component to provide an initial volume of aqueous media, wherein the filter has a membrane pore size from 0.07 to 0.45 μm; e) secondary filtration by cross-flow filtration to reduce the initial volume to provide a subsequent volume of aqueous media that is 10% to 90% of the initial volume, further wherein the cross-flow filtration is carried out with a process-scale tangential flow filter with a filtration area of 23 square feet or more, wherein all steps are carried out under aseptic conditions, f) the composition is prepared in quantities or batches greater than a liter; wherein the first emulsion is mixed in a first emulsification vessel of at least 10 liters in volume; and g) wherein the uniform size distribution has a number weighted mean particle size of at least 10 microns.

In some embodiments provided herein is a method of reducing an amount of a non-opioid analgesic administered to a subject following cesarean section surgery, the method comprising administering into the subject a composition comprising multivesicular liposomes comprising bupivacaine or a salt thereof and having a structure including multiple non-concentric chambers and comprising at least one amphipathic lipid and at least one neutral lipid, wherein said multivesicular liposomes are made by a process comprising removing organic solvent from multivesicular liposomes pre-droplets that comprise a first component core and an aqueous phase shell with an evaporation apparatus, the evaporation apparatus comprising: a solvent removal vessel having a top, a bottom and a circular wall; at least one atomizing nozzle; a carrier gas entrance orifice; a solvent removal gas exit orifice centrally connected to the top; and a product exit orifice connected to the bottom of the vessel,

wherein the process comprises: introducing the pre-droplets to the solvent removal vessel; applying a carrier gas in a tangential direction to the circular wall through the carrier gas entrance orifice; and removing a solvent removal gas through the solvent removal gas exit orifice to provide the large diameter synthetic membrane vesicles.

In some embodiments provided herein is a method of reducing a duration of time during which a non-opioid analgesic is administered to a subject following cesarean section surgery, the method comprising administering into the subject a pharmaceutical composition comprising: a) a multivesicular liposome comprising: at least one amphipathic lipid, and at least one neutral lipid; and b) an aqueous phase comprising: at least one polyhydroxy carboxylic acid and at least one di- or tri-protic mineral acid; and bupivacaine phosphate, wherein the aqueous phase is encapsulated within the multivesicular liposome.

In some embodiments provided herein is a method of reducing a duration of time during which a non-opioid analgesic is administered to a subject following cesarean section surgery, the method comprising administering into the subject a pharmaceutical composition comprising: a) a multivesicular liposome comprising: at least one amphipathic lipid, and at least one neutral lipid; and b) an aqueous phase comprising bupivacaine phosphate, wherein the aqueous phase is encapsulated within the multivesicular liposome.

In some embodiments provided herein is a method of reducing a duration of time during which a non-opioid analgesic is administered to a subject following cesarean section surgery, the method comprising administering into the subject a pharmaceutical composition comprising multivesicular liposomes encapsulating bupivacaine phosphate, said multivesicular liposomes comprising:

bupivacaine or a salt thereof;

phosphoric acid;

a lipid component comprising at least one amphipathic lipid and at least one neutral lipid lacking a hydrophilic head group; and,

optionally, a cholesterol and/or a plant sterol wherein said multivesicular liposomes are made by a process comprising:

a) preparing a first aqueous component comprising phosphoric acid;

b) preparing a lipid component comprising at least one organic solvent, at least one amphipathic lipid, and at least one neutral lipid lacking a hydrophilic head group;

c) mixing said first aqueous component and said lipid component to form a water-in-oil emulsion, wherein at least one component comprises bupivacaine or a salt thereof;

d) mixing said water-in-oil emulsion with a second aqueous component to form solvent spherules; and

e) removing the organic solvent from the solvent spherules to form multivesicular liposomes encapsulating bupivacaine phosphate.

In some embodiments provided herein is a method of reducing a duration of time during which a non-opioid analgesic is administered to a subject following cesarean section surgery, the method comprising administering into the subject a multivesicular liposomal particle pharmaceutical composition made by a process comprising: a) providing a volume of first emulsion by mixing a volume of a first aqueous phase and a volume of a volatile water-immiscible solvent phase, said solvent phase comprising at least one amphipathic lipid and at least one neutral lipid; b) mixing and emulsifying said first emulsion and a volume of a second aqueous phase in a high shear mixer to provide a volume of a second emulsion, said second emulsion comprising a continuous aqueous phase; and c) removing the volatile water-immiscible solvent from the second emulsion to form a volume of multivesicular liposomal particle composition, wherein said solvent removal comprises contacting the second emulsion with an inert gas flow; and wherein said process further comprises primary filtration of the multivesicular liposomal particle composition by cross-flow filtration using a filter having a membrane where the multivesicular liposomal particle composition does not pass through the membrane; wherein all steps are carried out under aseptic conditions, and wherein all solutions are sterile filtered, and wherein the multivesicular liposomal particle composition is immediately suitable for administration into humans; and wherein the primary filtration comprises: a first concentration of the multivesicular liposomal particle composition; and a buffer exchange, resulting in a pH of the multivesicular liposomal particle composition of between about 5 and about 8, and the primary filtration is conducted at a transmembrane pressure of from about 0.1 psi to about 20 psi, such as to about 7 psi.

In some embodiments provided herein is a method of reducing a duration of time during which a non-opioid analgesic is administered to a subject following cesarean section surgery, the method comprising administering into the subject a multivesicular liposomal particle pharmaceutical composition of pre-determined, uniform size distribution, made by a process comprising: a) providing a first emulsion by mixing a first aqueous phase and a volatile water-immiscible solvent phase, said solvent phase comprising at least one amphipathic lipid and at least one neutral lipid; b) mixing and emulsifying said first emulsion and a second aqueous phase in a mixer to provide a second emulsion, said second emulsion comprising a continuous aqueous phase; c) sparging the volatile water-immiscible solvent from the second emulsion to form an aqueous suspension of multivesicular liposomal particles by bubbling an inert gas through the second emulsion using at least one sparge ring, at least one sparge tube or at least one fit; d) primary filtration of the aqueous suspension of multivesicular liposomal particles by cross-flow filtration using a filter to exchange the second aqueous phase with an aqueous component to provide an initial volume of aqueous media, wherein the filter has a membrane pore size from 0.07 to 0.45 μm; e) secondary filtration by cross-flow filtration to reduce the initial volume to provide a subsequent volume of aqueous media that is 10% to 90% of the initial volume, further wherein the cross-flow filtration is carried out with a process-scale tangential flow filter with a filtration area of 23 square feet or more, wherein all steps are carried out under aseptic conditions, f) the composition is prepared in quantities or batches greater than a liter; wherein the first emulsion is mixed in a first emulsification vessel of at least 10 liters in volume; and g) wherein the uniform size distribution has a number weighted mean particle size of at least 10 microns.

In some embodiments provided herein is a method of reducing a duration of time during which a non-opioid analgesic is administered to a subject following cesarean section surgery, the method comprising administering into the subject a composition comprising multivesicular liposomes comprising bupivacaine or a salt thereof and having a structure including multiple non-concentric chambers and comprising at least one amphipathic lipid and at least one neutral lipid, wherein said multivesicular liposomes are made by a process comprising removing organic solvent from multivesicular liposomes pre-droplets that comprise a first component core and an aqueous phase shell with an evaporation apparatus, the evaporation apparatus comprising: a solvent removal vessel having a top, a bottom and a circular wall; at least one atomizing nozzle; a carrier gas entrance orifice; a solvent removal gas exit orifice centrally connected to the top; and a product exit orifice connected to the bottom of the vessel,

wherein the process comprises: introducing the pre-droplets to the solvent removal vessel; applying a carrier gas in a tangential direction to the circular wall through the carrier gas entrance orifice; and removing a solvent removal gas through the solvent removal gas exit orifice to provide the large diameter synthetic membrane vesicles.

In some embodiments the at least one polyhydroxy carboxylic acid is selected from the group consisting of glucuronic acid, gluconic acid and tartaric acid.

In some embodiments the amphipathic lipid is selected from the group consisting of phosphatidylcholines, phosphatidylethanolamines, sphingomyelins, lysophosphatidylcholines, lysophosphatidylethanolamines, phosphatidylglycerols, phosphatidylserines, phosphatidylinositols, phosphatidic acids, cardiolipins, diacyl dimethylammonium propanes, and stearylamines.

In some embodiments the neutral lipid comprises at least one triglyceride.

In some embodiments the method comprises administering a therapeutically effective amount of the pharmaceutical composition.

In some embodiments the pharmaceutical composition comprises a therapeutically effective amount of bupivacaine phosphate. In some embodiments wherein the pharmaceutical composition comprises bupivacaine phosphate in an amount equivalent to from about 20 mg to about 300 mg of bupivacaine. In some embodiments the pharmaceutical composition comprises an amount equivalent to from about 133 mg to about 266 mg of bupivacaine.

In some embodiments the pharmaceutical composition comprises an amount equivalent to from about 10 mg to about 300 mg of bupivacaine. In some embodiments the pharmaceutical composition comprises an amount equivalent to from about 133 mg to about 266 mg of bupivacaine. In some embodiments the pharmaceutical composition comprises an amount equivalent to from about 10 mg to about 70 mg of bupivacaine. In some embodiments the pharmaceutical composition comprises an amount equivalent to from about 20 mg to about 60 mg of bupivacaine. In some embodiments the pharmaceutical composition comprises an amount equivalent to from about 20 mg to about 50 mg of bupivacaine. In some embodiments the pharmaceutical composition comprises an amount equivalent to from about 20 mg to about 40 mg of bupivacaine. In some embodiments the pharmaceutical composition comprises an amount equivalent to from about 20 mg to about 30 mg of bupivacaine. In some embodiments the pharmaceutical composition comprises an amount equivalent to from about 30 mg to about 60 mg of bupivacaine. In some embodiments the pharmaceutical composition comprises an amount equivalent to from about 30 mg to about 50 mg of bupivacaine. In some embodiments the pharmaceutical composition comprises an amount equivalent to from about 30 mg to about 40 mg of bupivacaine. In some embodiments the amount of the pharmaceutical composition described herein is equivalent to from about 10 mg to about 70 mg of bupivacaine. In some embodiments the amount of the pharmaceutical composition described herein is equivalent to from about 10 mg to about 60 mg of bupivacaine. In some embodiments the amount of the pharmaceutical composition described herein is equivalent to from about 20 mg to about 60 mg of bupivacaine. In some embodiments the amount of the pharmaceutical composition described herein is equivalent to from about 20 mg to about 50 mg of bupivacaine. In some embodiments the amount of the pharmaceutical composition described herein is equivalent to from about 20 mg to about 40 mg of bupivacaine. In some embodiments the amount of the pharmaceutical composition described herein is equivalent to from about 20 mg to about 30 mg of bupivacaine. In some embodiments the amount of the pharmaceutical composition described herein is equivalent to from about 30 mg to about 60 mg of bupivacaine. In some embodiments the amount of the pharmaceutical composition described herein is equivalent to from about 30 mg to about 50 mg of bupivacaine. In some embodiments the amount of the pharmaceutical composition described herein is equivalent to from about 30 mg to about 40 mg of bupivacaine.

In some embodiments the method comprises administering the pharmaceutical composition by epidural injection.

In some embodiments the method does not comprise administering the pharmaceutical composition by epidural injection.

In some embodiments the method comprises administering the pharmaceutical composition by transversus abdominis plane (TAP) block.

In some embodiments method comprises administering the pharmaceutical composition following completion of the cesarean section surgery.

In some embodiments method comprises administering the pharmaceutical composition prior to the cesarean section surgery.

In some embodiments method comprises administering the pharmaceutical composition during the cesarean section surgery.

In some embodiments the method comprises administering the pharmaceutical composition by transversus abdominis plane (TAP) block under ultrasound guidance.

In some embodiments the method comprises administering an opioid to the subject following completion of the cesarean section surgery.

In some embodiments of the methods herein, the opioid is administered in a total amount less than 200 mg, such as less than 100 mg, such as less than 50 mg, such as less than 25 mg, such as less than 15 mg, in the first about 72 hours following completion of the cesarean section surgery. In some embodiments, the opioid is oxycodone and the method comprises administering oxycodone in a total amount less than 15 mg.in the first about 72 hours following completion of the cesarean section surgery.

In some embodiments, the method comprises administering one or more morphinans to the subject. In some embodiments, the method comprises administering morphine to the subject. In some more particular embodiments, the morphine is administered to the subject for up to 72 hours following completion of the cesarean section surgery.

In some embodiments, the method comprises administering one or more analgesics to the subject, such as one or more NSAIDs to the subject. In some embodiments, the method comprises administering one or more of ketorolac, acetaminophen or ibuprofen to the subject. In some embodiments, the method comprises administering two or more of ketorolac, acetaminophen or ibuprofen to the subject. In some embodiments, the method comprises administering ketorolac, acetaminophen and ibuprofen to the subject. In some more particular embodiments, the analgesic, such as the NSAID, such as the one or more of ketorolac, acetaminophen or ibuprofen, is administered to the subject for up to 72 hours following completion of the cesarean section surgery.

In some embodiments, the subject has an AUC for VAS pain intensity scores over the first 72 hours following completion of the cesarean section surgery of from about 100 to about 200, such as about 125 to 175, such as about 140 to 160, such as about 150, such as about 147.9.

In some embodiments of the methods herein, the subject has a distress from itchiness score as determined by the OBAS scale of less than 4, such as 0, 1, 2 or 3.

In some embodiments of the methods herein, the plasma Cmax of bupivacaine in the subject is about 150 ng/mL to about 250 ng/mL, such as about 175 ng/mL to about 225 ng/mL, such as about 200 ng/mL, such as about 210 mg/mL, for an amount of the pharmaceutical composition described herein that is equivalent to about 133 mg of bupivacaine. In some embodiments, the Cmax occurs after about 48 hours following completion of the cesarean section surgery. In some embodiments, the Cmax occurs after about 72 hours following completion of the cesarean section surgery.

In some embodiments of the methods herein, the plasma Cmax of bupivacaine in the subject is about 300 ng/mL to about 550 ng/mL, such as about 350 ng/mL to about 500 ng/mL, such as about 450 mg/mL, such as about 460 ng/mL, for an amount of the pharmaceutical composition described herein that is equivalent to about 266 mg of bupivacaine. In some embodiments, the Cmax occurs after about 48 hours following completion of the cesarean section surgery. In some embodiments, the Cmax occurs after about 72 hours following completion of the cesarean section surgery.

In some embodiments of the methods herein, the plasma Cmax of bupivacaine in the subject is less than about 850 ng/mL, such as less than about 800 ng/mL, such as less than about 750 ng/mL, such as less than about 700 ng/mL, such as less than about 650 ng/mL, such as less than about 600 ng/mL.

In some embodiments, the subject is a female human.

In some embodiments of the methods herein, the subject is a first subject, wherein in the first about 72 hours following completion of the cesarean section surgery the opioid is administered to the first subject in a total amount that is lower than a total amount of an opioid that is administered to a second subject in the first about 72 hours following completion of a cesarean section surgery in the second subject, wherein a pharmaceutical composition comprising: a) a multivesicular liposome comprising: at least one amphipathic lipid, and at least one neutral lipid; and b) an aqueous phase comprising: at least one polyhydroxy carboxylic acid and at least one di- or tri-protic mineral acid; and bupivacaine phosphate, wherein the aqueous phase is encapsulated within the multivesicular liposome, is not administered to the second subject.

In some embodiments of the methods herein, the subject is a first subject, wherein in the first about 72 hours following completion of the cesarean section surgery the opioid is administered to the first subject in a total amount that is lower than a total amount of an opioid that is administered to a second subject in the first about 72 hours following completion of a cesarean section surgery in the second subject, wherein a pharmaceutical composition comprising: a) a multivesicular liposome comprising: at least one amphipathic lipid, and at least one neutral lipid; and b) an aqueous phase comprising: at least one polyhydroxy carboxylic acid and at least one di- or tri-protic mineral acid; and bupivacaine phosphate, wherein the aqueous phase is encapsulated within the multivesicular liposome, is not administered to the second subject.

In some embodiments of the methods herein, the subject is a first subject, wherein in the first about 72 hours following completion of the cesarean section surgery the opioid is administered to the first subject in a total amount that is lower than a total amount of an opioid that is administered to a second subject in the first about 72 hours following completion of a cesarean section surgery in the second subject, wherein a pharmaceutical composition comprising multivesicular liposomes encapsulating bupivacaine phosphate, said multivesicular liposomes comprising:

bupivacaine or a salt thereof;

phosphoric acid;

a lipid component comprising at least one amphipathic lipid and at least one neutral lipid lacking a hydrophilic head group; and,

optionally, a cholesterol and/or a plant sterol wherein said multivesicular liposomes are made by a process comprising:

a) preparing a first aqueous component comprising phosphoric acid;

b) preparing a lipid component comprising at least one organic solvent, at least one amphipathic lipid, and at least one neutral lipid lacking a hydrophilic head group;

c) mixing said first aqueous component and said lipid component to form a water-in-oil emulsion, wherein at least one component comprises bupivacaine or a salt thereof;

d) mixing said water-in-oil emulsion with a second aqueous component to form solvent spherules; and

e) removing the organic solvent from the solvent spherules to form multivesicular liposomes encapsulating bupivacaine phosphate,

is not administered to the second subject.

In some embodiments of the methods herein, the subject is a first subject, wherein in the first about 72 hours following completion of the cesarean section surgery the opioid is administered to the first subject in a total amount that is lower than a total amount of an opioid that is administered to a second subject in the first about 72 hours following completion of a cesarean section surgery in the second subject,

wherein a multivesicular liposomal particle pharmaceutical composition made by a process comprising: a) providing a volume of first emulsion by mixing a volume of a first aqueous phase and a volume of a volatile water-immiscible solvent phase, said solvent phase comprising at least one amphipathic lipid and at least one neutral lipid; b) mixing and emulsifying said first emulsion and a volume of a second aqueous phase in a high shear mixer to provide a volume of a second emulsion, said second emulsion comprising a continuous aqueous phase; and c) removing the volatile water-immiscible solvent from the second emulsion to form a volume of multivesicular liposomal particle composition, wherein said solvent removal comprises contacting the second emulsion with an inert gas flow; and wherein said process further comprises primary filtration of the multivesicular liposomal particle composition by cross-flow filtration using a filter having a membrane where the multivesicular liposomal particle composition does not pass through the membrane; wherein all steps are carried out under aseptic conditions, and wherein all solutions are sterile filtered, and wherein the multivesicular liposomal particle composition is immediately suitable for administration into humans; and wherein the primary filtration comprises: a first concentration of the multivesicular liposomal particle composition; and a buffer exchange, resulting in a pH of the multivesicular liposomal particle composition of between about 5 and about 8, and the primary filtration is conducted at a transmembrane pressure of from about 0.1 psi to about 20 psi, such as to about 7 psi,

is not administered to the second subject.

In some embodiments of the methods herein, the subject is a first subject, wherein in the first about 72 hours following completion of the cesarean section surgery the opioid is administered to the first subject in a total amount that is lower than a total amount of an opioid that is administered to a second subject in the first about 72 hours following completion of a cesarean section surgery in the second subject,

wherein a multivesicular liposomal particle pharmaceutical composition of pre-determined, uniform size distribution, made by a process comprising: a) providing a first emulsion by mixing a first aqueous phase and a volatile water-immiscible solvent phase, said solvent phase comprising at least one amphipathic lipid and at least one neutral lipid; b) mixing and emulsifying said first emulsion and a second aqueous phase in a mixer to provide a second emulsion, said second emulsion comprising a continuous aqueous phase; c) sparging the volatile water-immiscible solvent from the second emulsion to form an aqueous suspension of multivesicular liposomal particles by bubbling an inert gas through the second emulsion using at least one sparge ring, at least one sparge tube or at least one fit; d) primary filtration of the aqueous suspension of multivesicular liposomal particles by cross-flow filtration using a filter to exchange the second aqueous phase with an aqueous component to provide an initial volume of aqueous media, wherein the filter has a membrane pore size from 0.07 to 0.45 μm; e) secondary filtration by cross-flow filtration to reduce the initial volume to provide a subsequent volume of aqueous media that is 10% to 90% of the initial volume, further wherein the cross-flow filtration is carried out with a process-scale tangential flow filter with a filtration area of 23 square feet or more, wherein all steps are carried out under aseptic conditions, f) the composition is prepared in quantities or batches greater than a liter; wherein the first emulsion is mixed in a first emulsification vessel of at least 10 liters in volume; and g) wherein the uniform size distribution has a number weighted mean particle size of at least 10 microns,

is not administered to the second subject.

In some embodiments of the methods herein, the subject is a first subject, wherein in the first about 72 hours following completion of the cesarean section surgery the opioid is administered to the first subject in a total amount that is lower than a total amount of an opioid that is administered to a second subject in the first about 72 hours following completion of a cesarean section surgery in the second subject, wherein a composition comprising multivesicular liposomes comprising bupivacaine or a salt thereof and having a structure including multiple non-concentric chambers and comprising at least one amphipathic lipid and at least one neutral lipid, wherein said multivesicular liposomes are made by a process comprising removing organic solvent from multivesicular liposomes pre-droplets that comprise a first component core and an aqueous phase shell with an evaporation apparatus, the evaporation apparatus comprising: a solvent removal vessel having a top, a bottom and a circular wall; at least one atomizing nozzle; a carrier gas entrance orifice; a solvent removal gas exit orifice centrally connected to the top; and a product exit orifice connected to the bottom of the vessel,

wherein the process comprises: introducing the pre-droplets to the solvent removal vessel; applying a carrier gas in a tangential direction to the circular wall through the carrier gas entrance orifice; and removing a solvent removal gas through the solvent removal gas exit orifice to provide the large diameter synthetic membrane vesicles,

is not administered to the second subject.

In some embodiments, the opioid that is administered to the first subject and the opioid that is administered to the second subject are the same. In some embodiments, the opioid that is administered to the first subject and the opioid that is administered to the second subject are different.

In some embodiments of the methods herein, the subject is a first subject, wherein in the first about 72 hours following completion of the cesarean section surgery the opioid is administered to the first subject in a total amount that is lower than a total amount of an opioid that is administered to each of a plurality of second subjects in the first about 72 hours following respective completion of a cesarean section surgery in each of the second subjects, wherein a pharmaceutical composition comprising: a) a multivesicular liposome comprising: at least one amphipathic lipid, and at least one neutral lipid; and b) an aqueous phase comprising: at least one polyhydroxy carboxylic acid and at least one di- or tri-protic mineral acid; and bupivacaine phosphate, wherein the aqueous phase is encapsulated within the multivesicular liposome, is not administered to the second subjects.

In some embodiments of the methods herein, the subject is a first subject, wherein in the first about 72 hours following completion of the cesarean section surgery the opioid is administered to the first subject in a total amount that is lower than a total amount of an opioid that is administered to each of a plurality of second subjects in the first about 72 hours following respective completion of a cesarean section surgery in each of the second subjects, wherein a pharmaceutical composition comprising: a) a multivesicular liposome comprising: at least one amphipathic lipid, and at least one neutral lipid; and b) an aqueous phase comprising: at least one polyhydroxy carboxylic acid and at least one di- or tri-protic mineral acid; and bupivacaine phosphate, wherein the aqueous phase is encapsulated within the multivesicular liposome, is not administered to the second subjects.

In some embodiments of the methods herein, the subject is a first subject, wherein in the first about 72 hours following completion of the cesarean section surgery the opioid is administered to the first subject in a total amount that is lower than a total amount of an opioid that is administered to each of a plurality of second subjects in the first about 72 hours following respective completion of a cesarean section surgery in each of the second subjects, wherein a pharmaceutical composition comprising multivesicular liposomes encapsulating bupivacaine phosphate, said multivesicular liposomes comprising:

bupivacaine or a salt thereof;

phosphoric acid;

a lipid component comprising at least one amphipathic lipid and at least one neutral lipid lacking a hydrophilic head group; and,

optionally, a cholesterol and/or a plant sterol wherein said multivesicular liposomes are made by a process comprising:

a) preparing a first aqueous component comprising phosphoric acid;

b) preparing a lipid component comprising at least one organic solvent, at least one amphipathic lipid, and at least one neutral lipid lacking a hydrophilic head group;

c) mixing said first aqueous component and said lipid component to form a water-in-oil emulsion, wherein at least one component comprises bupivacaine or a salt thereof;

d) mixing said water-in-oil emulsion with a second aqueous component to form solvent spherules; and

e) removing the organic solvent from the solvent spherules to form multivesicular liposomes encapsulating bupivacaine phosphate,

is not administered to the second subjects.

In some embodiments of the methods herein, the subject is a first subject, wherein in the first about 72 hours following completion of the cesarean section surgery the opioid is administered to the first subject in a total amount that is lower than a total amount of an opioid that is administered to each of a plurality of second subjects in the first about 72 hours following respective completion of a cesarean section surgery in each of the second subjects, wherein a multivesicular liposomal particle pharmaceutical composition made by a process comprising: a) providing a volume of first emulsion by mixing a volume of a first aqueous phase and a volume of a volatile water-immiscible solvent phase, said solvent phase comprising at least one amphipathic lipid and at least one neutral lipid; b) mixing and emulsifying said first emulsion and a volume of a second aqueous phase in a high shear mixer to provide a volume of a second emulsion, said second emulsion comprising a continuous aqueous phase; and c) removing the volatile water-immiscible solvent from the second emulsion to form a volume of multivesicular liposomal particle composition, wherein said solvent removal comprises contacting the second emulsion with an inert gas flow; and wherein said process further comprises primary filtration of the multivesicular liposomal particle composition by cross-flow filtration using a filter having a membrane where the multivesicular liposomal particle composition does not pass through the membrane; wherein all steps are carried out under aseptic conditions, and wherein all solutions are sterile filtered, and wherein the multivesicular liposomal particle composition is immediately suitable for administration into humans; and wherein the primary filtration comprises: a first concentration of the multivesicular liposomal particle composition; and a buffer exchange, resulting in a pH of the multivesicular liposomal particle composition of between about 5 and about 8, and the primary filtration is conducted at a transmembrane pressure of from about 0.1 psi to about 20 psi, such as to about 7 psi,

is not administered to the second subjects.

In some embodiments of the methods herein, the subject is a first subject, wherein in the first about 72 hours following completion of the cesarean section surgery the opioid is administered to the first subject in a total amount that is lower than a total amount of an opioid that is administered to each of a plurality of second subjects in the first about 72 hours following respective completion of a cesarean section surgery in each of the second subjects,

wherein a multivesicular liposomal particle pharmaceutical composition of pre-determined, uniform size distribution, made by a process comprising: a) providing a first emulsion by mixing a first aqueous phase and a volatile water-immiscible solvent phase, said solvent phase comprising at least one amphipathic lipid and at least one neutral lipid; b) mixing and emulsifying said first emulsion and a second aqueous phase in a mixer to provide a second emulsion, said second emulsion comprising a continuous aqueous phase; c) sparging the volatile water-immiscible solvent from the second emulsion to form an aqueous suspension of multivesicular liposomal particles by bubbling an inert gas through the second emulsion using at least one sparge ring, at least one sparge tube or at least one fit; d) primary filtration of the aqueous suspension of multivesicular liposomal particles by cross-flow filtration using a filter to exchange the second aqueous phase with an aqueous component to provide an initial volume of aqueous media, wherein the filter has a membrane pore size from 0.07 to 0.45 μm; e) secondary filtration by cross-flow filtration to reduce the initial volume to provide a subsequent volume of aqueous media that is 10% to 90% of the initial volume, further wherein the cross-flow filtration is carried out with a process-scale tangential flow filter with a filtration area of 23 square feet or more, wherein all steps are carried out under aseptic conditions, f) the composition is prepared in quantities or batches greater than a liter; wherein the first emulsion is mixed in a first emulsification vessel of at least 10 liters in volume; and g) wherein the uniform size distribution has a number weighted mean particle size of at least 10 microns,

is not administered to the second subjects.

In some embodiments of the methods herein, the subject is a first subject, wherein in the first about 72 hours following completion of the cesarean section surgery the opioid is administered to the first subject in a total amount that is lower than a total amount of an opioid that is administered to each of a plurality of second subjects in the first about 72 hours following respective completion of a cesarean section surgery in each of the second subjects,

wherein a composition comprising multivesicular liposomes comprising bupivacaine or a salt thereof and having a structure including multiple non-concentric chambers and comprising at least one amphipathic lipid and at least one neutral lipid, wherein said multivesicular liposomes are made by a process comprising removing organic solvent from multivesicular liposomes pre-droplets that comprise a first component core and an aqueous phase shell with an evaporation apparatus, the evaporation apparatus comprising: a solvent removal vessel having a top, a bottom and a circular wall; at least one atomizing nozzle; a carrier gas entrance orifice; a solvent removal gas exit orifice centrally connected to the top; and a product exit orifice connected to the bottom of the vessel,

wherein the process comprises: introducing the pre-droplets to the solvent removal vessel; applying a carrier gas in a tangential direction to the circular wall through the carrier gas entrance orifice; and removing a solvent removal gas through the solvent removal gas exit orifice to provide the large diameter synthetic membrane vesicles,

is not administered to the second subjects.

In some embodiments, the opioid that is administered to the first subject and the opioid that is administered to at least one of the second subjects are the same. In some embodiments, the opioid that is administered to the first subject and the opioid that is administered to at least one of the second subjects are different. In some embodiments, the opioid that is administered to the first subject and the opioids that are administered to the second subjects are the same. In some embodiments, the opioid that is administered to the first subject and the opioids that are administered to the second subjects are different. In some embodiments, the total amount of an opioid that is administered to each of a plurality of second subjects is a mean total amount.

In some embodiments of the methods herein, non-liposomal bupivacaine or a salt thereof, such as bupivacaine hydrochloride, is administered to the second subject following completion of the cesarean section surgery in the second subject.

In some embodiments of the methods herein, the opioid is administered to the first subject in a total amount that is at least about 20% lower, such as at least about 30% lower, such as at least about 40% lower, such as at least about 50% lower than the total amount of the opioid that is administered to the second subject. In some embodiments of the methods herein, the opioid is administered to the first subject in a total amount that is up to 100% lower (that is, no opioid is administered to the first subject), such as up to 90% lower, such as up to 80% lower, such as up to 70% lower, such as up to 60% lower, than the total amount of the opioid that is administered to the second subject.

In some embodiments of the methods herein, in the first about 72 hours following completion of the cesarean section surgery in the first subject the opioid is administered to the first subject in a total amount that is at least about 20% lower, such as at least about 30% lower, such as at least about 40% lower, such as at least about 50% lower than the total amount of the opioid that is administered to the second subject in the first about 72 hours following completion of the cesarean section surgery in the second subject. In some embodiments of the methods herein, the opioid is administered to the first subject in a total amount that is up to 100% lower (that is, no opioid is administered to the first subject), such as up to 90% lower, such as up to 80% lower, such as up to 70% lower, such as up to 60% lower, than the total amount of the opioid that is administered to the second subject.

In some embodiments of the methods herein, in the first about 24 hours following completion of the cesarean section surgery in the first subject the opioid is administered to the first subject in a total amount that is lower than the total amount of an opioid that is administered to the second subject in the first about 24 hours following completion of the cesarean section surgery in the second subject. In some embodiments of the methods herein, in the first about 24 hours following completion of the cesarean section surgery in the first subject the opioid is administered to the first subject in a total amount that is at least about 20% lower, such as at least about 30% lower, such as at least about 40% lower, such as at least about 50% lower than the total amount of the opioid that is administered to the second subject in the first about 24 hours following completion of the cesarean section surgery in the second subject. In some embodiments of the methods herein, the opioid is administered to the first subject in a total amount that is up to 100% lower (that is, no opioid is administered to the first subject), such as up to 90% lower, such as up to 80% lower, such as up to 70% lower, such as up to 60% lower, than the total amount of the opioid that is administered to the second subject.

In some embodiments of the methods herein, in the first about 48 hours following completion of the cesarean section surgery in the first subject the opioid is administered to the first subject in a total amount that is lower than the total amount of an opioid that is administered to the second subject in the first about 48 hours following completion of the cesarean section surgery in the second subject. In some embodiments of the methods herein, in the first about 48 hours following completion of the cesarean section surgery in the first subject the opioid is administered to the first subject in a total amount that is at least about 20% lower, such as at least about 30% lower, such as at least about 40% lower, such as at least about 50% lower than the total amount of the opioid that is administered to the second subject in the first about 48 hours following completion of the cesarean section surgery in the second subject. In some embodiments of the methods herein, the opioid is administered to the first subject in a total amount that is up to 100% lower (that is, no opioid is administered to the first subject), such as up to 90% lower, such as up to 80% lower, such as up to 70% lower, such as up to 60% lower, than the total amount of the opioid that is administered to the second subject.

In some embodiments of the methods herein, in the first about 7 days following completion of the cesarean section surgery in the first subject the opioid is administered to the first subject in a total amount that is lower than the total amount of an opioid that is administered to the second subject in the first about 7 days following completion of the cesarean section surgery in the second subject. In some embodiments of the methods herein, in the first about 7 days following completion of the cesarean section surgery in the first subject the opioid is administered to the first subject in a total amount that is at least about 20% lower, such as at least about 30% lower, such as at least about 40% lower, such as at least about 50% lower than the total amount of the opioid that is administered to the second subject in the first about 7 days following completion of the cesarean section surgery in the second subject. In some embodiments of the methods herein, the opioid is administered to the first subject in a total amount that is up to 100% lower (that is, no opioid is administered to the first subject), such as up to 90% lower, such as up to 80% lower, such as up to 70% lower, such as up to 60% lower, than the total amount of the opioid that is administered to the second subject.

In some embodiments of the methods herein, in the first about 14 days following completion of the cesarean section surgery in the first subject the opioid is administered to the first subject in a total amount that is lower than the total amount of an opioid that is administered to the second subject in the first about 14 days following completion of the cesarean section surgery in the second subject. In some embodiments of the methods herein, in the first about 14 days following completion of the cesarean section surgery in the first subject the opioid is administered to the first subject in a total amount that is at least about 20% lower, such as at least about 30% lower, such as at least about 40% lower, such as at least about 50% lower than the total amount of the opioid that is administered to the second subject in the first about 14 days following completion of the cesarean section surgery in the second subject. In some embodiments of the methods herein, the opioid is administered to the first subject in a total amount that is up to 100% lower (that is, no opioid is administered to the first subject), such as up to 90% lower, such as up to 80% lower, such as up to 70% lower, such as up to 60% lower, than the total amount of the opioid that is administered to the second subject.

In some embodiments, the opioid that is administered to the first subject and the opioid that is administered to the second subject are the same. In some embodiments, the opioid that is administered to the first subject and the opioid that is administered to the second subject are different.

In some embodiments of the methods herein, the first subject has an AUC for VAS pain intensity scores over the first 72 hours following completion of the cesarean section surgery in the first subject of from about 100 to about 200, such as about 125 to 175, such as about 140 to 160, such as about 150, such as about 147.9; and the second subject has an AUC for VAS pain intensity scores over the first 72 hours following completion of the cesarean section surgery in the second subject of from about 150 to about 250, such as about 165 to 200, such as about 170 to 190, such as about 175 to 180, such as about 178.5.

In some embodiments of the methods herein, the first subject has an AUC for VAS pain intensity scores over the first 72 hours following completion of the cesarean section surgery in the first subject that is at least about 10% lower, such as at least about 17% lower, such as about 27% to about 25% lower, such as at least about 25% lower, than the AUC for VAS pain intensity scores for the second subject over the first 72 hours following completion of the cesarean section surgery in the second subject. In some embodiments the first subject has an AUC for VAS pain intensity scores over the first 72 hours following completion of the cesarean section surgery in the first subject that is up to 100% lower (that is, the AUC is 0), such as up to 90% lower, such as up to 80% lower, such as up to 70% lower, such as up to 60% lower, than the AUC for VAS pain intensity scores for the second subject.

In some embodiments of the methods herein, the first subject has a distress from itchiness score as determined by the OBAS scale that is lower than for the second subject.

In some embodiments of the methods herein, the plasma concentration of bupivacaine in the first subject after about 120 hours following completion of the cesarean section surgery in the first subject is at least about 10%, such as least about 20% higher , such as at least about 30% higher , such as at least about 40% higher , such as 50% higher than the plasma concentration of bupivacaine in the second subject after about 120 hours following completion of the cesarean section surgery in the second subject. In some embodiments, the plasma concentration is up to 500% higher, such as up to 400% higher, such as up to 300% higher, such as up to 200% higher, such as up to 100% higher, than the plasma concentration in the second subject.

In some embodiments, the method does not comprise administering an opioid to the subject following completion of the cesarean section.

In some embodiments, the method does not comprise administering one or more morphinans to the subject. In some embodiments, the method does not comprise administering morphine to the subject.

In some embodiments, the method does not comprise administering an opioid to the first subject following completion of the cesarean section.

In some embodiments, the method does not comprise administering one or more morphinans to the first subject. In some embodiments, the method does not comprise administering morphine to the first subject.

In some embodiments, the method comprises administering into the subject an amount of the pharmaceutical composition described herein that is equivalent to about 20 to about 300 mg of bupivacaine. In some embodiments, the method comprises administering into the subject an amount of the pharmaceutical composition described herein that is equivalent to about 100 to about 300 mg of bupivacaine. In some embodiments, the method comprises administering into the subject an amount of the pharmaceutical composition described herein that is equivalent to about 133 to about 300 mg of bupivacaine. In some embodiments, the method comprises administering into the subject an amount of the pharmaceutical composition described herein that is equivalent to about 133 to about 266 mg of bupivacaine. In some embodiments, the method comprises administering into the subject about 20 mL of the pharmaceutical composition described herein containing about 266 mg of bupivacaine.

In some embodiments, the method comprises administering into the first subject an amount of the pharmaceutical composition described herein that is equivalent to about 20 to about 300 mg of bupivacaine. In some embodiments, the method comprises administering into the first subject an amount of the pharmaceutical composition described herein that is equivalent to about 100 to about 300 mg of bupivacaine. In some embodiments, the method comprises administering into the first subject an amount of the pharmaceutical composition described herein that is equivalent to about 133 to about 300 mg of bupivacaine. In some embodiments, the method comprises administering into the first subject an amount of the pharmaceutical composition described herein that is equivalent to about 133 to about 266 mg of bupivacaine. In some embodiments, the method comprises administering into the first subject about 20 mL of the pharmaceutical composition described herein containing about 266 mg of bupivacaine.

In some embodiments, the method comprises administering into the subject the pharmaceutical composition comprising bupivacaine phosphate in an amount equivalent to 266 mg of bupivacaine and non-liposomal bupivacaine in an amount equivalent to 44 mg of bupivacaine base.

In some embodiments, the method comprises administering into the first subject the pharmaceutical composition comprising bupivacaine phosphate in an amount equivalent to 266 mg of bupivacaine and non-liposomal bupivacaine in an amount equivalent to 44 mg of bupivacaine, and administering into the second subject non-liposomal bupivacaine in an amount equivalent to 44 mg of bupivacaine.

In some embodiments the non-liposomal bupivacaine may be, for example, in the form of the hydrochloride salt of bupivacaine.

In some embodiments, the method comprises administering one or more non-opioid analgesics to the subject. In some embodiments, the one or more non-opioid analgesics are one or more NSAIDs. In some embodiments, the one or more non-opioid analgesics are one or more of ketorolac, acetaminophen or ibuprofen. Thus, in some embodiments, the method comprises administering one or more of ketorolac, acetaminophen or ibuprofen to the subject, wherein the one or more of ketorolac, acetaminophen or ibuprofen, is administered to the subject for up to 72 hours following completion of the cesarean section surgery in the following amounts:
IV ketorolac 15 mg once at the time of skin incision closure and prior to the TAP infiltration
Intravenous (IV) acetaminophen 1000 mcg at the time of skin incision closure
Scheduled oral (PO) acetaminophen 650 mg at the end of surgery and every 6 hours (q6 h) for up to 72 hours
Scheduled PO ibuprofen 600 mg at the end of surgery and q6 h for up to 72 hours

In some embodiments, the method comprises administering an opioid to a subject, wherein one or more opioids are administered in the following amounts:
oral immediate-release oxycodone at 5-10 mg every 4 hours or as needed
IV morphine at 1-2 mg or hydromorphone initiated at 0.3-0.5 mg every 4 hours or as needed

In some embodiments, the first subject is a female human and the second subject is a female human.

In some embodiments of any of the methods disclosed herein, the method produces postsurgical local analgesia.

In some embodiments of any of the methods disclosed herein, the method produces postsurgical regional analgesia.

Unless otherwise defined, all technical and scientific terms used herein have the same meaning as commonly understood by one of ordinary skill in the art to which this invention belongs. Although methods and materials similar to those described herein can be used in the practice or testing of the present invention, suitable methods and materials are described below. All publications, patent applications, patents, and other references mentioned herein are incorporated by reference in their entirety. In addition, the materials, methods, and examples are illustrative only and not intended to be limiting.

The term “therapeutically effective” as it pertains to bupivacaine or a salt thereof, such as bupivacaine phosphate, present in the pharmaceutical compositions described herein, means that an anesthetic present in the first aqueous phase within the multivesicular liposome is released in a manner sufficient to achieve a particular level of anesthesia. Exact dosages will vary depending on such factors as the particular anesthetic, as well as patient factors such as age, sex, general condition, and the like. Those of skill in the art can readily take these factors into account and use them to establish effective therapeutic concentrations without resort to undue experimentation.

As used herein, “non-liposomal bupivacaine” refers to bupivacaine or a salt thereof that is not in liposomal form. For example, “non-liposomal bupivacaine” refers to bupivacaine or a salt thereof that is not comprised in a multivesicular liposome. The term “non-liposomal bupivacaine” encompasses compositions comprising bupivacaine, or a salt thereof, that is not in liposomal form.

As used herein, “completion” of the cesarean section surgery occurs when skin incision closure is complete.

As used herein, a “VAS pain intensity score” refers to the Visual Analog Scale pain intensity score described in Delgado et al., J Am Acad Orthop Surg Glob Res Rev. 2018 Mar; 2(3): e088, published online 2018 Mar. 23. doi: 10.5435/JAAOSGlobal-D-17-00088, incorporated by reference herein in its entirety.

The compositions used in the methods disclosed herein comprise a) a multivesicular liposome comprising: at least one amphipathic lipid, and at least one neutral lipid; and b) an aqueous phase comprising: at least one polyhydroxy carboxylic acid and at least one di- or tri-protic mineral acid; and bupivacaine phosphate, wherein the aqueous phase is encapsulated within the multivesicular liposome.

In some embodiments the aqueous phase further comprises hydrochloric acid.

Multivesicular liposomes (or “MVL”, which is used herein to refer to a multivesicular liposome or a plurality of multivesicular liposomes) are lipid vesicles having multiple non-concentric internal aqueous chambers having internal membranes distributed as a network throughout the MVL. The chambers may contain acids which are effective to enable the encapsulation of bupivacaine or a salt thereof and to modulate its release rate. A preparation of MVL is described, for example, in Kim et al., Biochim. Biophys. Acta 728, 339-348, 1983. In some embodiments, a MVL is prepared in accordance with a process as described in U.S. Pat. No. 9,192,575, incorporated by reference herein in its entirety.

### Multivesicular Liposomes

The anesthetic compositions of the invention also include multivesicular liposomes (MVL) which encapsulate and provide modulated and sustained release of the anesthetics described above. The MVL are made by the following process. A “water-in-oil” type emulsion containing a non-hydrohalic acid salt of any of the anesthetics described above is formed from two immiscible phases, a lipid phase and a first aqueous phase.

The lipid phase is made up of at least one amphipathic lipid and at least one neutral lipid in a volatile organic solvent. The term “amphipathic lipid” refers to molecules having a hydrophilic “head” group and a hydrophobic “tail” group and may have membrane-forming capability. As used herein, amphipathic lipids include those having a net negative charge, a net positive charge, and zwitterionic lipids (having no net charge at their isoelectric point). The term “neutral lipid” refers to oils or fats that have no vesicle-forming capability by themselves, and lack a charged or hydrophilic “head” group. Examples of neutral lipids include, but are not limited to, glycerol esters, glycol esters, tocopherol esters, sterol esters which lack a charged or hydrophilic “head” group, and alkanes and squalenes.

The amphipathic lipid is chosen from a wide range of lipids having a hydrophobic region and a hydrophilic region in the same molecule. Suitable amphipathic lipids are zwitterionic phospholipids, including phosphatidylcholine, phosphatidylethanolamines, sphingomyelins, lysophosphatidylcholines, and lysophosphatidylethanolamines. Also suitable are the anionic amphipathic phospholipids such as phosphatidylglycerols, phosphatidylserines, phosphatidylinositols, phosphatidic acids, and cardiolipins. Also suitable are the cationic amphipathic lipids such as acyl trimethylammonium propanes, diacyl dimethylammonium propanes, and stearylamine.

Suitable neutral lipids are triglycerides, propylene glycol esters, ethylene glycol esters, and squalene. Examples of triglycerides useful in the present invention are triolein, tripalmitolein, trimyristolein, trilinolein, tributyrin, tricaproin, tricaprylin, and tricaprin. The fatty chains in the triglycerides useful in the present invention can be all the same, or not all the same (mixed chain triglycerides), including all different. Both saturated and unsaturated fatty chains are useful in the present invention. The propylene glycol esters can be mixed diesters of caprylic and capric acids.

Many types of volatile organic solvents can be used in the present invention, including ethers, esters, halogenated ethers, hydrocarbons, halohydrocarbons, or Freons. For example, diethyl ether, chloroform, tetrahydrofuran, ethyl acetate, Forane, and any combinations thereof are suitable for use in making the anesthetic compositions of the present invention.

Optionally, but highly desirably, other components are included in the lipid phase. Among these are cholesterol or plant sterols.

The first aqueous phase includes an anesthetic, at least one polyhydroxy carboxylic acid, and at least one di- or tri-protic mineral acid. In some embodiments of the invention, also included is hydrochloric acid. Hydrochloric acid is not an essential constituent, but rather is optional and desirable in some embodiments. The di- or tri-protic mineral acids include sulfuric acid, and phosphoric acid. Also included in the first aqueous phase are such polyhydroxy carboxylic acids as glucuronic acid, gluconic acid, and tartaric acid. The di- and tri-protic mineral acids and the polyhydroxy organic acids are present in the first aqueous phase in concentrations of from 0.01 mM to about 0.5 M, or preferably from about 5 mM to about 300 mM. When hydrochloric acid is used, it is present in lower amounts, from about 0.1 mM to about 50 mM, or preferably from about 0.5 mM to about 25 mM.

The lipid phase and first aqueous phase are mixed by mechanical turbulence, such as through use of rotating or vibrating blades, shaking, extrusion through baffled structures or porous pipes, by ultrasound, or by nozzle atomization, to produce a water-in-oil emulsion. Thus, the anesthetics of the invention are encapsulated directly in the first step of MVL manufacture.

The whole water-in-oil emulsion is then dispersed into a second aqueous phase by means described above, to form solvent spherules suspended in the second aqueous phase. The term “solvent spherules” refers to a microscopic spheroid droplet of organic solvent, within which are suspended multiple smaller droplets of aqueous solution. The resulting solvent spherules therefore contain multiple aqueous droplets with the anesthetic dissolved therein. The second aqueous phase can contain additional components such as glucose, and/or lysine.

The volatile organic solvent is then removed from the spherules, for instance by surface evaporation from the suspension: When the solvent is substantially or completely evaporated, MVL are formed. Gases which can be used for the evaporation include nitrogen, argon, helium, oxygen, hydrogen, and carbon dioxide. Alternatively, the volatile solvent can be removed by sparging, rotary evaporation, or with the use of solvent selective membranes.

In some embodiments, a MVL is prepared in accordance with a process as described in U.S. Pat. No. 8,182,835, incorporated by reference herein in its entirety. In some embodiments, a MVL is prepared in accordance with a process as described in U.S. Pat. No. 8,834,921, incorporated by reference herein in its entirety. In some embodiments, a MVL is prepared in accordance with a process as described in U.S. Pat. No. 9,205,052, incorporated by reference herein in its entirety.

In some embodiments the multivesicular liposomes (“MVL”) are made by the following process. A “water-in-oil” type emulsion containing a non-hydrohalic acid salt of bupivacaine, such as bupivacaine phosphate, is formed from two immiscible phases, a lipid phase and a first aqueous phase. The lipid phase is made up of at least one amphipathic lipid and at least one neutral lipid in a volatile organic solvent. The term “amphipathic lipid” refers to molecules having a hydrophilic “head” group and a hydrophobic “tail” group and may have membrane-forming capability. As used herein, amphipathic lipids include those having a net negative charge, a net positive charge, and zwitterionic lipids (having no net charge at their isoelectric point). The term “neutral lipid” refers to oils or fats that have no vesicle-forming capability by themselves, and lack a charged or hydrophilic “head” group. Examples of neutral lipids include, but are not limited to, glycerol esters, glycol esters, tocopherol esters, sterol esters which lack a charged or hydrophilic “head” group, and alkanes and squalenes.

The amphipathic lipid is chosen from a wide range of lipids having a hydrophobic region and a hydrophilic region in the same molecule. Suitable amphipathic lipids are zwitterionic phospholipids, including phosphatidylcholines, phosphatidylethanolamines, sphingomyelins, lysophosphatidylcholines, and lysophosphatidylethanolamines. Also suitable are the anionic amphipathic phospholipids such as phosphatidylglycerols, phosphatidylserines, phosphatidylinositols, phosphatidic acids, and cardiolipins. Also suitable are the cationic amphipathic lipids such as acyl trimethylammonium propanes, diacyl dimethylammonium propanes, and stearylamines.

Suitable neutral lipids are triglycerides, propylene glycol esters, ethylene glycol esters, and squalene. Examples of triglycerides useful in the present invention are triolein, tripalmitolein, trimyristolein, trilinolein, tributyrin, tricaproin, tricaprylin, and tricaprin. The fatty chains in the triglycerides useful in the present invention can be all the same, or not all the same (mixed chain triglycerides), including all different. Both saturated and unsaturated fatty chains are useful in the present invention. The propylene glycol esters can be mixed diesters of caprylic and capric acids.

Many types of volatile organic solvents can be used in the present invention, including ethers, esters, halogenated ethers, hydrocarbons, halohydrocarbons, or Freons. For example, diethyl ether, chloroform, tetrahydrofuran, ethyl acetate, Forane, and any combinations thereof are suitable for use in making the compositions of the present invention.

Optionally, other components are included in the lipid phase. Among these are cholesterol or plant sterols.

The first aqueous phase includes bupivacaine or a salt thereof, such as bupivacaine phosphate, at least one polyhydroxy carboxylic acid, and at least one di- or tri-protic mineral acid. In some embodiments, also included is hydrochloric acid. The di- or tri-protic mineral acids include sulfuric acid, and phosphoric acid. Also included in the first aqueous phase are such polyhydroxy carboxylic acids as glucuronic acid, gluconic acid, and tartaric acid. The di- and tri-protic mineral acids and the polyhydroxy organic acids are present in the first aqueous phase in concentrations of from 0.01 mM to about 0.5 M, or preferably from about 5 mM to about 300 mM. When hydrochloric acid is used, it is present in lower amounts, from about 0.1 mM to about 50 mM, or preferably from about 0.5 mM to about 25 mM.

The lipid phase and first aqueous phase are mixed by mechanical turbulence, such as through use of rotating or vibrating blades, shaking, extrusion through baffled structures or porous pipes, by ultrasound, or by nozzle atomization, to produce a water-in-oil emulsion. Thus, bupivacaine or a salt thereof, such as bupivacaine phosphate, is encapsulated directly in the first step of MVL manufacture.

The whole water-in-oil emulsion is then dispersed into a second aqueous phase by means described above, to form solvent spherules suspended in the second aqueous phase. The term “solvent spherules” refers to a microscopic spheroid droplet of organic solvent, within which are suspended multiple smaller droplets of aqueous solution. The resulting solvent spherules therefore contain multiple aqueous droplets with the bupivacaine or a salt thereof, such as bupivacaine phosphate, dissolved therein. The second aqueous phase can contain additional components such as glucose, and/or lysine.

The volatile organic solvent is then removed from the spherules, for instance by surface evaporation from the suspension: When the solvent is substantially or completely evaporated, MVL are formed. Gases which can be used for the evaporation include nitrogen, argon, helium, oxygen, hydrogen, and carbon dioxide. Alternatively, the volatile solvent can be removed by sparging, rotary evaporation, or with the use of solvent selective membranes.

In some embodiments, an MVL is prepared in accordance with a process as described in U.S. Pat. No. 10,398,648, incorporated by reference herein in its entirety. In some embodiments, a MVL is prepared in accordance with a process as described in US 9,585,838 incorporated by reference herein in its entirety.

In some embodiments, a MVL is prepared in accordance with a process as described in US 2011-0250264, US 2013-0306759, US 2013-0177634, US 2013-0177633, US 2013-0177635, US 2013-0195965, US 2013-0177636, US 2013-0183373, US 2013-0177638, US 2013-0177637, US 2013-0183372, US 2013-0183375, US 2016-0361260 or US 2018-0092847, each of which is incorporated by reference herein in its entirety.

### EXAMPLES

### Example 1—Clinical Trial

A multicenter, randomized, double-blind clinical trial examining efficacy and safety of the pharmaceutical composition comprising: a) the multivesicular liposome disclosed herein and b) the aqueous phase disclosed herein, wherein the aqueous phase is encapsulated within the multivesicular liposome, was performed as described below. The pharmaceutical composition is referred to below as the “multivesicular liposomal” pharmaceutical composition, or “MVL”.The trial compared opioid consumption after TAP block with MVL plus BUPI HCl versus TAP block with BUPI HCl alone as part of a multimodal analgesia protocol including intrathecal morphine in patients undergoing elective cesarean section surgery with spinal anesthesia. The trial also determined pain-related scores such as VAS and/or OBAS. The trial also determined the level of pruritus in subjects to whom opioids were administered following the cesarean section.

Glossary of Terms

AE adverse event

ANCOVA analysis of covariance

AUC area under the curve

AUC₀₋₇₂ area under the curve through 72 hours

BUPI bupivacaine, not in liposomal form—also referred to as non-liposomal bupivacaine

CI confidence interval

HCl hydrochloride

LAST local anesthetic systemic toxicity

MVL multivesicular liposomal bupivacaine

LSM least squares mean

MED morphine equivalent dosing

mITT modified intent-to-treat

NSAID nonsteroidal anti-inflammatory drug

OBAS overall benefit of analgesia score

SE standard error

TAP transversus abdominis plane

TEAE treatment-emergent adverse event

VAS visual analog scale

Methods:

Women (n=186) with term pregnancies of 37 to 42 weeks of gestation undergoing elective cesarean delivery under spinal anesthesia including 150 μg intrathecal morphine were randomized (1:1) to TAP block with MVL 266 mg plus bupivacaine HCl 50 mg or bupivacaine HCl alone. Efficacy was evaluated in a prespecified modified intent-to-treat population of those who met the study protocol criteria. The primary endpoint was total postsurgical opioid consumption (oral morphine equivalent dosing [MED]) through 72 hours. Pain intensity was measured using a visual analog scale. Treatment-emergent adverse events (TEAEs) were recorded through day 14. Exclusion criteria included high-risk pregnancies, a pregnancy-induced medical condition or complication, ≥3 prior cesarean deliveries, pre-pregnancy body mass index >50 kg/m², concurrent painful physical condition that may require analgesia (including opioids), and history of illicit drug use. Patients were excluded if the epidural component of the combined spinal epidural anesthetic was used. Because BUPI is regarded as compatible with breastfeeding by the World Health Organization, there were no restrictions regarding the inclusion of women who wished to breastfeed following treatment.

Eligible patients were randomized in a blinded 1:1 ratio to receive TAP infiltration with MVL plus BUPI HCl or active BUPI HCl alone. Prior to surgery, all patients received spinal or combined spinal epidural anesthesia with 1.4 to 1.6 mL of hyperbaric BUPI HCl 0.75% with 150 μg of morphine and 15 μg of fentanyl. While a narrow range of hyperbaric BUPI HCl was specified, the expected duration of action for this amount of BUPI HCl in spinal anesthesia is ˜2 hours. Thus, it is believed that any small differences in volume would not have significant effects on any outcomes beyond 24 hours. Intraoperatively, ketamine and midazolam were permitted if clinically indicated; dexamethasone use was prohibited. At the time of skin incision closure, 15 mg of intravenous ketorolac and 1000 mg of intravenous acetaminophen were administered. Within 90 minutes of the completion of the cesarean section surgery, a bilateral, 2-point classic TAP block with MVL 266 mg plus BUPI HCl 50 mg or BUPI HCl 50 mg alone (30 mL each side; 60 mL total for each) was performed under ultrasound guidance by experienced physicians, primarily staff anesthesiologists. Subcutaneous lidocaine could be administered at the area of the needle insertion site if needed for patient comfort. The TAP infiltration included 2 steps; TAP needle placement and saline hydrodissection followed by study drug mixture infiltration into the TAP. Confirmatory ultrasound images were taken after the needle position had been established and after infiltration. Beginning 6 hours after skin incision closure, 650 mg of oral acetaminophen and 600 mg of oral ibuprofen were given every 6 hours for up to 72 hours or until hospital discharge. Opioid pain medication to treat postsurgical pain was permitted if requested by the patient and consisted of immediate-release oxycodone initiated at 5-10 mg every 4 hours or as needed. If unable to tolerate or initial rescue medication failure was experienced, intravenous morphine initiated at 1-2 mg or hydromorphone initiated at 0.3-0.5 mg could be administered every 4 hours or as needed. Patients remained in the hospital for up to 72 hours after surgery. Follow-up calls for safety purposes and to assess for pain were scheduled for all patients on postsurgical day 14.

A centralized randomization system was used to generate treatment assignments. Patients and research personnel collecting data were blinded to treatment group allocation; only designated unblinded pharmacists responsible for preparing study drugs received the unblinded randomization assignments.

End Points

The primary end point was total postsurgical opioid consumption through 72 hours. Secondary end points were total postsurgical opioid consumption through 24 hours, 48 hours, 1 week, and 2 weeks; time to first postsurgical opioid rescue medication; area under the curve (AUC) of visual analog scale (VAS) pain intensity scores through 72 hours (AUC₀₋₇₂); the percentage of opioid-free patients through 72 hours; and the percentage of opioid-spared patients through 72 hours. Opioid-free was defined as not receiving any opioid medication postoperatively. Opioid-spared was defined a priori as taking ≤10 mg of oxycodone (or equivalent) postoperatively with an overall benefit of analgesia score (OBAS) of 0 for OBAS survey questions 2 through 6.

Tertiary efficacy end points included AUC of the VAS pain intensity scores at various time points. Safety end points were assessed by incidence of treatment-emergent AEs (TEAEs) from the time of administration of study drug (which occurred after skin incision closure) through day 14. Relationship of AEs to either drug injected was assessed by the investigator.

Statistical Analysis

The safety analysis set included all patients who received TAP block with MVL plus BUPI HCl or BUPI HCl alone. Safety analyses were based on actual treatment received. Efficacy was evaluated in a modified intent-to-treat (mITT) analysis set that was defined a priori and prespecified to include all randomized patients assessed for safety who underwent cesarean delivery and who also met the prespecified study criteria for correct TAP placement, local anesthetic dosing, and adherence to a multimodal postsurgical analgesic regimen. Patients who otherwise had protocol deviations beyond these prespecified criteria were included in the mITT efficacy analysis. Efficacy was analyzed on the basis of randomized treatment regardless of the treatment received. The ultrasound images for TAP placement for each patient were adjudicated blindly by an independent review committee of expert anesthesiologists to assess if the TAP block was placed correctly. Two independent reviewers were assigned for the review of each patient. If the reviewers disagreed, a third reviewer was assigned to make the final determination. An additional post hoc efficacy analysis was performed in all patients who received TAP block with MVL plus BUPI HCl or BUPI HCl alone.

Statistical analyses of study end points were prespecified in the statistical analysis plan. For the primary end point, comparison between treatment groups was assessed using an analysis of covariance (ANCOVA) model with treatment and site as the main effects and age and height as covariates. Additionally, the percent reduction in total postsurgical oral morphine equivalent dosing (MED) was determined on the basis of the least squares mean (LSM) for each group estimated from the ANCOVA model. No multiplicity adjustments were made for efficacy.

AUC₀₋₇₂ for VAS pain intensity was analyzed using an ANCOVA model with treatment and site as main effects and age and height as covariates to determine if MVL plus BUPI was noninferior to BUPI alone. The LSMs for treatment difference and 95% confidence intervals (CIs) were used in a step-down statistical approach. To test for noninferiority, a margin of 36 was set on the upper bound of the 95% CI. This noninferiority margin was set on the basis of an average pain intensity score difference of 0.5, which was considered a clinically nonsignificant difference over 72 hours and is a smaller margin than what has been reported in previous studies. If noninferiority was determined, then superiority was tested. If the upper bound of the 95% CI was <0, then superiority was determined. If superiority was determined, then the 1-sided superiority test P value is shown. If the superiority test failed, only the 1-sided noninferiority test P value is shown. This methodology was used for AUC of the VAS pain intensity scores at all-time points, with noninferiority margins corresponding to an average pain intensity score difference of 0.5 over the respective time interval.

The percentage of opioid-spared and opioid-free patients was analyzed using a logistic regression model with treatment, site, age, and height as explanatory variables. Time to first opioid rescue was calculated from the time of the end of surgery to first opioid medication and analyzed using a Cox regression model with treatment and site as factors and age and height as covariates.

Sample size determination for this study was based on results of a previous retrospective study of TAP infiltration with MVL plus BUPI in women undergoing cesarean delivery. The coefficient of variation in that study was ˜60%. Assuming a log-normal distribution for total opioid consumption with a 60% coefficient of variation, 5% alpha level, 1:1 randomization ratio, and 80% power, 72 patients per treatment arm would be sufficient to detect a 30% difference using a 2-sample t-test. Assuming 5% of the patients were not evaluable, a total sample size of ˜152 patients was needed to ensure 144 evaluable individuals.

Results

Study Cohort

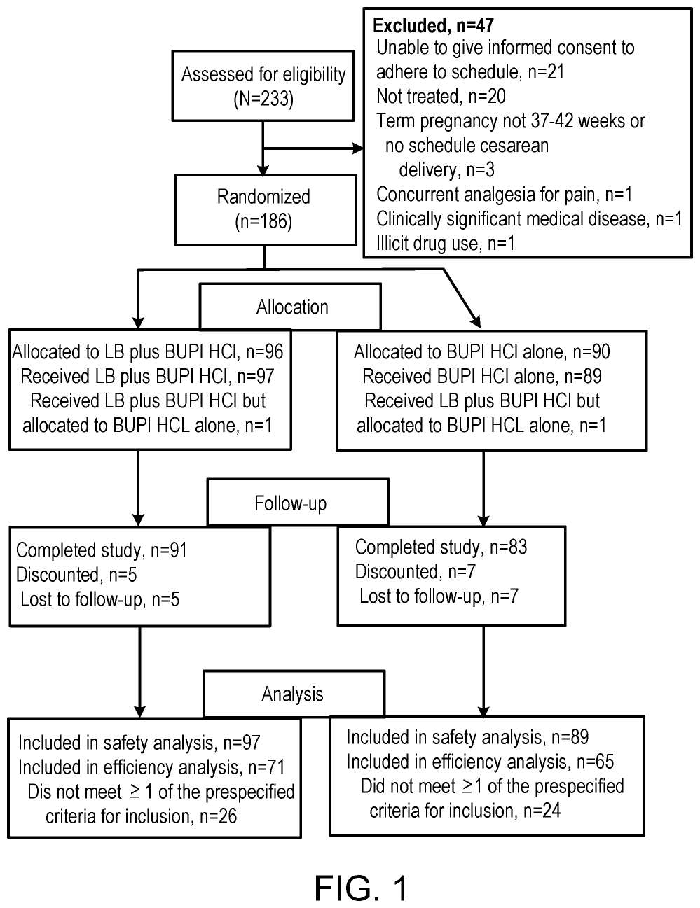

A total of 233 patients were screened for eligibility in the study, with 186 (MVL plus BUPI HCl, n=96; BUPI HCl alone, n=90) being randomized to study drugs (FIG. 1). Of these, 136 patients (MVL plus BUPI HCl, n=71; BUPI HCl alone, n=65) met criteria for inclusion in the mITT efficacy analysis set. A total of 50 patients (MVL plus BUPI HCl, n=26; BUPI HCl alone, n=24) were not included in the mITT efficacy analysis set because they did not meet ≥1 criteria for correct TAP placement (n=13), local anesthetic dosing (n=12), or multimodal postsurgical analgesic regimen (n=39). One patient was randomized to BUPI HCl alone but received MVL 266 mg plus BUPI HCl; this patient was included in the MVL plus BUPI HCl safety analysis set and the BUPI HCl alone mITT efficacy analysis set. Patient demographics and baseline characteristics were comparable across treatment groups (Table 1).

Primary Efficacy End Point

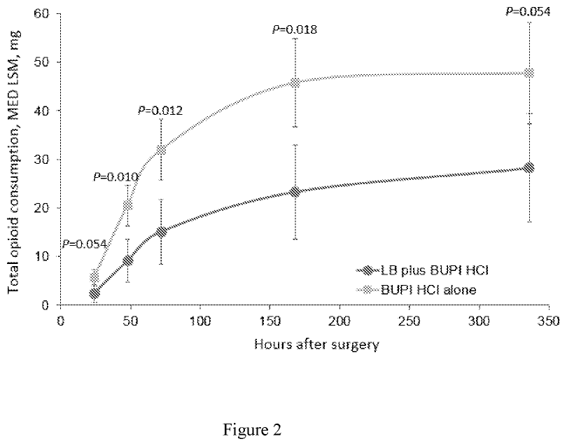

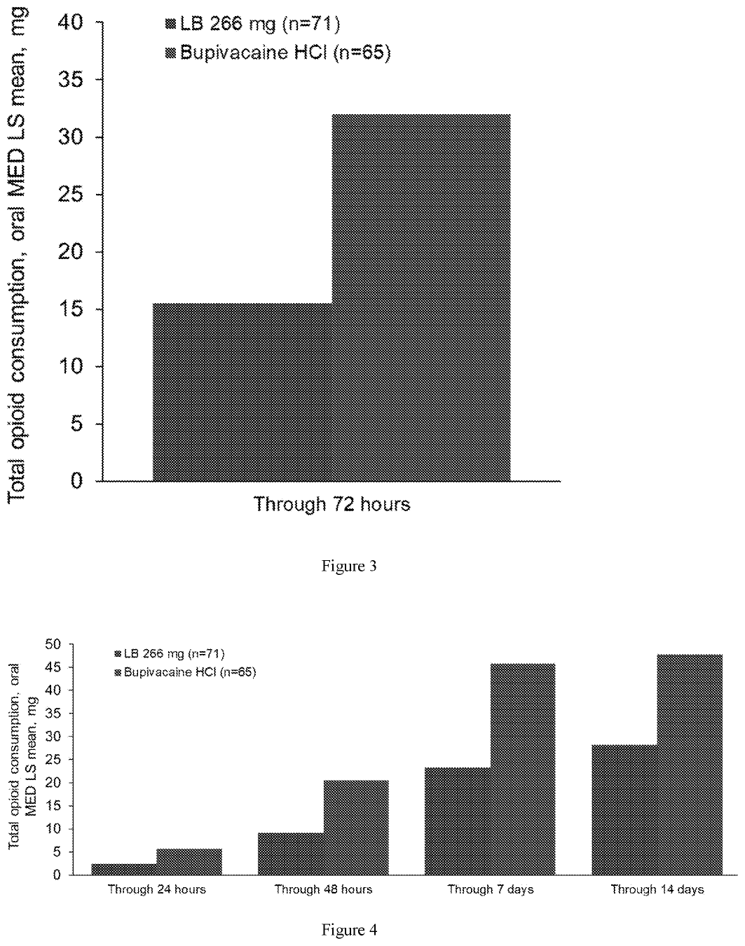

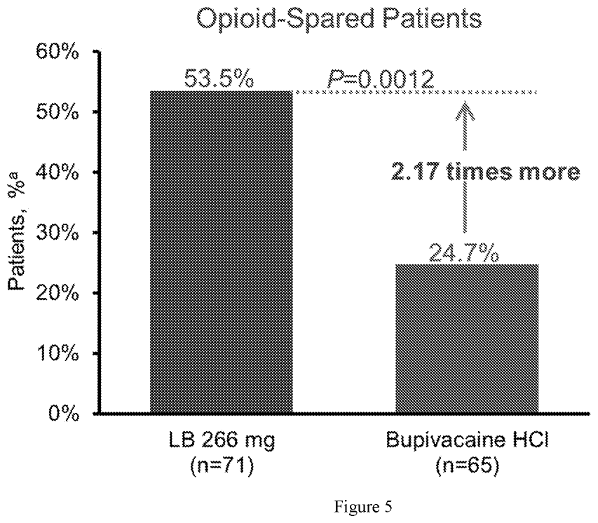

The primary end point of the study was met; total opioid consumption in MED through 72 hours was reduced by 51.6% in the MVL plus BUPI HCl group versus that in the BUPI HCl alone group (LSM [standard error (SE)], 15.5 [6.67] vs 32.0 [6.25] mg; FIG. 2. This corresponded to an LSM treatment difference in MED of −16.5 mg (95% CI, −30.8, −2.2 mg; P=0.0117).

Secondary and Tertiary Efficacy End Points

In the MVL plus BUPI HCl group versus the BUPI HCl alone group, significant reductions in opioid consumption were observed at 48 hours (LSM [SE], 9.1 [4.46] vs 20.5 [4.18] MED mg; P=0.0096) and 7 days (LSM [SE], 23.3 [9.75] vs 45.8 [9.13] MED mg; P=0.0175), A numerical reduction in opioid consumption was observed at 14 days (LSM [SE], 28.2 [11.20] vs 47.8 [10.49] MED mg; P=0.0542).

The percentage of opioid-spared patients was 2.2 times higher in the MVL plus BUPI HCl group versus that in the BUPI HCl alone group at 72 hours (53.5% vs 24.7%; P=0.0012). The time to first opioid rescue ranged from 2.3 to 345.2 hours in the MVL plus BUPI HCl group and from 2.5 to 345.7 hours in the BUPI HCl alone group. The median time to first opioid rescue was longer in the MVL plus BUPI HCl group (53.2 hours) than that in the BUPI HCl alone group (41.1 hours); however, this difference was not statistically significant (P=0.7536). The percentage of opioid-free patients was not different in the MVL plus BUPI HCl group versus that in the BUPI HCl alone group at 24 hours (95.6% vs 93.7%; P=0.1752), 48 hours (58.4% vs 50.6%; P=0.1961), and 72 hours (51.9% vs 48.6%; P=0.3609).

The LSM (SE) AUC₀₋₇₂ of VAS pain intensity scores through 72 hours was 147.9 (21.13) in the MVL plus BUPI HCl group vs 178.5 (19.78) in the BUPI HCl alone group. The LSM treatment difference between the groups was −30.6 (95% CI, −75.9, 14.7), with the upper limit meeting the prespecified noninferiority margin of 36 (P=0.0020), but not the superiority margin of 0. Reductions in AUC of the VAS pain intensity scores were observed in the MVL plus BUPI HCl group at most time intervals examined from 12 hours through the 72-hour period (e.g., 12-24, 24-36, and 36-48 hours), with all meeting the criteria for noninferiority, but not superiority.

The summed pain intensity scores through 72 hours were lower in the MVL plus BUPI HCl group versus those in the BUPI HCl alone group meeting the criteria for noninferiority as prespecified. An analysis of the integrated rank assessment using sum of pain intensity scores at rest and MED of opioid rescue medications through 48 hours showed a statistically significant treatment difference (P=0.0398). No significant differences were observed between treatment groups for other tertiary end points.

In the post hoc efficacy analysis of the primary end point with all patients treated, which included those who were excluded from the mITT analysis set because they did not meet criteria for correct TAP blocks, local anesthetic dosing, and multimodal postsurgical analgesic regimen, there were no significant differences in LSM (SE) total opioid consumption (MED) through 72 hours in the MVL plus BUPI HCl group (24.3 [6.03] mg) and the BUPI HCl alone group (27.5 [5.84] mg; P=0.3122). Similarly, there were no significant differences in the LSM (SE) AUC₀₋₇₂ of VAS pain intensity scores between the MVL plus BUPI HCl group (158.8 [19.1]) and BUPI HCl alone group (168.3 [18.4]; LSM treatment difference, −9.6 [95% CI, −49.2, 30.0]).

Safety

The table below provides a summary of TEAEs and of treatment-related TEAEs:

TABLE
Treatment-Emergent Adverse Events
(Overall and Treatment-Related; Safety Analysis Set)

| | MVL + BUPI | BUPI |
|---|---|---|
| | HCl | alone |
| | (n = 97) | (n = 89) |
| Any TEAE | 62 (63.9) | 50 (56.2) |
| Any treatment-related TEAE | 6 (6.2) | 9 (10.1) |
| Serious TEAE | 3 (3.1) | 3 (3.4) |
| Fatal TEAE | 0 (0.0) | 0 (0.0) |

TEAEs occurring in >5% of patients in either group

| | | |
|---|---|---|
| Pruritus | 27 (27.8) | 28 (31.5) |
| Nausea | 24 (24.7) | 11 (12.4) |
| Vomiting | 12 (12.4) | 6 (6.7) |
| Headache | 6 (6.2) | 10 (11.2) |
| Dizziness | 6 (6.2) | 5 (5.6) |
| Constipation | 6 (6.2) | 4 (4.5) |
| Backpain | 3 (3.1) | 5 (5.6) |
| Rash | 5 (5.2) | 3 (3.4) |

Treatment-related TEAEs occurring in patients in either group^{a}

| | | |
|---|---|---|
| Pruritus | 2 (2.1) | 8 (9.0) |
| Nausea | 3 (3.1) | 0 (0.0) |
| Vomiting | 3 (3.1) | 0 (0.0) |
| Dizziness | 1 (1.0) | 0 (0.0) |
| Backpain | 0 (0.0) | 1 (1.1) |
| Dysuria | 0 (0.0) | 1 (1.1) |

Values are the number (percentage). All AEs were recorded through day 14. TEAEs were recorded on or after the administration of study drug (which occurred after skin incision closure) through day 14.
^{a}Treatment-relatedness was determined by the investigator. AE, adverse event; BUPI, bupivacaine; HCl, hydrochloride; MVL, multivesicular liposomal bupivacaine; TEAE, treatment-emergent AE.

Summary of Results:

Total opioid consumption through 72 hours was reduced with MVL plus bupivacaine HCl versus bupivacaine HCl alone (least squares mean [LSM] [standard error (SE)] MED, 15.5 [6.67] vs 32.0 [6.25] mg). This corresponded to an LSM treatment difference of −16.5 mg (95% confidence interval [CI], −30.8, −2.2 mg); P=0.0117), and the primary endpoint was met. A significant reduction in overall opioid consumption was also observed with MVL plus bupivacaine HCl versus bupivacaine HCl alone at 1 week (LSM [SE], 23.3 [9.75] vs 45.8 [9.13] mg; P=0.0175). The area under the curve of imputed pain intensity scores through 72 hours supported non-inferiority of MVL plus bupivacaine HCl versus bupivacaine HCl alone (LSM [SE], 147.9 [21.13] vs 178.5 [19.78]; LSM treatment difference, −30.6 [95% CI, −75.9, 14.7]). In the MVL plus bupivacaine HCl group, 63.6% of patients experienced a TEAE versus 56.2% in the bupivacaine HCl alone group. The most common TEAEs were pruritus and nausea. Serious TEAEs were rare (˜3% in both groups) with no fatal TEAEs.

Conclusions:

TAP block using MVL plus bupivacaine HCl as part of a multimodal analgesia protocol incorporating intrathecal morphine resulted in reduced opioid consumption after cesarean delivery. Results suggest an opioid-reducing benefit of adding MVL to bupivacaine TAP blocks for cesarean delivery.

### Example 2—Clinical Trial

A Multicenter, Randomized, Active-Controlled Study to Evaluate the Efficacy and Safety of EXPAREL When Administered via Infiltration into the Transversus Abdominis Plane versus Standard of Care in Subjects Undergoing Elective Cesarean Section (CHOICE)

Objectives

Primary objective: To compare total opioid consumption through 72 hours following EXPAREL infiltration into the transversus abdominis plane (TAP) with standard of care (SOC) in subjects undergoing an elective cesarean section (C-section).

Secondary objective: To assess efficacy and safety parameters and participant satisfaction.

Medications

Pre-operative medications: Use of pre-operative analgesics (e.g., opioid medications, Tylenol® [acetaminophen], non-steroidal anti-inflammatory drugs [NSAIDs]) is prohibited.
Group 1: 150 mcg Duramorph® (SOC arm). No EXPAREL TAP infiltration following skin-incision closure.
   Subjects randomized to Group 1 will receive an intrathecal injection of 150 mcg preservative-free morphine for spinal injection (e.g., Duramorph) in conjunction with single-shot spinal anesthesia using 1.4 to 1.6 mL bupivacaine hydrochloride (HCl) 0.75% and 15 mcg fentanyl. Postoperative pain management will follow the multimodal pain regimen as defined in this protocol.
Group 2: 50 mcg Duramorph+EXPAREL TAP infiltration following skin-incision closure+postoperative multimodal pain regimen as defined in this protocol.
   Subjects randomized to Group 2 will receive an intrathecal injection of 50 mcg preservative-free morphine for spinal injection (e.g., Duramorph) in conjunction with single-shot spinal anesthesia using 1.4 to 1.6 mL bupivacaine HCl 0.75%. If deemed necessary by the investigator, intrathecal fentanyl can be given prior to incision for the C-section. If intrathecal fentanyl is used, the dose of 15 mcg fentanyl, date, and time of the usage must be recorded. In addition, Group 2 subjects will receive EXPAREL TAP infiltration following skin-incision closure plus postoperative multimodal pain regimen.
Group 3: EXPAREL TAP infiltration following skin-incision closure+postoperative multimodal pain regimen as defined in this protocol. No Duramorph.
   Subjects randomized to Group 3 will NOT receive Duramorph as a spinal injection. This group will receive single-shot spinal anesthesia using 1.4 to 1.6 mL bupivacaine HCl 0.75%. If deemed necessary by the investigator, intrathecal fentanyl can be given prior to incision for the C-section. If intrathecal fentanyl is used, the dose of 15 mcg fentanyl, date, and time of the usage must be recorded. In addition, Group 3 subjects will receive EXPAREL

In all treatment groups, a combined spinal epidural (CSE) anesthesia technique may also be used provided the epidural component is not used. Subjects who receive the epidural component of the CSE anesthesia must be immediately withdrawn from the study.

Intraoperative medications: The intraoperative use of ketamine and midazolam (Versed®) is discouraged but may be permitted if clinically indicated based on the investigator's discretion (all medications must be appropriately recorded [i.e., drug, dose, and route of administration]). Prophylactic use of dexamethasone for prevention of nausea and vomiting is prohibited.

For Groups 2 and 3, after delivery of the baby and prior to the TAP infiltration, a small amount of lidocaine (<2 mL) may be administered subcutaneously to form a skin wheal over the area of the needle insertion site. A 2-point classic TAP block, in 2 steps (see Pharmacy Binder), must be performed under ultrasound guidance and must be performed no more than 90 minutes after skin-incision closure of the C-section. A confirmatory ultrasound image or video will be taken of each side of the abdomen after the TAP needle position has been established and following infiltration of study drug.

TAP infiltration (Groups 2 and 3): The TAP infiltration includes two steps: (1) TAP needle placement and saline hydrodissection and (2) study drug mixture infiltration into the TAP. Each step must be performed on one side of the abdomen and must be repeated on the contralateral side to complete the bilateral, 2-point TAP required for the study. For complete, step-by-step instructions on performing the TAP infiltration under ultrasound guidance, please refer to the Pharmacy Binder.

Postsurgical Analgesia: Participant-controlled analgesia is not permitted.

The following multimodal pain regimens will be used for all treatment groups. The date, time, and dose of all standardized multimodal pain medications administered must be recorded.

All Subjects in Groups 1, 2, and 3:

At the time of skin-incision closure:
Intravenous (IV) ketorolac 30 mg (1 dose)
IV acetaminophen 1000 mg (1 dose)

Beginning 6 hours after skin-incision closure:
IV ketorolac 30 mg beginning 6 hours from the administration of IV ketorolac at the time of skin-incision closure and then every 6 hours (q6 h) for the next 18 hours (i.e., total of 120 mg as four 30-mg doses over first 24 hours from the time of skin-incision closure)
IV acetaminophen 1000 mg beginning 6 hours from the administration of IV acetaminophen at the time of skin-incision closure and then every 6 hours (q6 h) for the next 18 hours (i.e., total of 4000 mg as four 1000-mg doses over first 24 hours from the time of skin-incision closure)
Scheduled oral (PO) Tylenol® (acetaminophen) 975 mg beginning 6 hours from the administration of the last dose of IV acetaminophen and q6 h for up to 72 hours or hospital discharge (whichever occurs first)
Scheduled PO ibuprofen 600 mg beginning 6 hours from the administration of the last dose of IV ketorolac and then q6 h for up to 72 hours or hospital discharge (whichever occurs first)

This multimodal pain regimen is a requirement for all subjects in the study and is not subject to investigator discretion. The date, time, and dose of all standardized multimodal pain medications administered must be recorded. Note: The scheduled PO medication will be administered on a q6 h schedule only till hospital discharge.

Rescue Medication: When breakthrough pain occurs, the study staff or the floor nurses on duty will need to ensure that the study subject is strictly following the protocol-specific multimodal pain regimen schedule (q6 h as defined in the protocol) before considering the use of the opioids as a rescue, especially in cases where the visual analog scale (VAS) score is relatively low. Subjects should receive opioid rescue pain medication only upon request for breakthrough pain.

Postsurgical rescue medication will comprise PO immediate-release oxycodone (initiated at 5 mg as needed [PRN]). If the 5 mg oxycodone is not sufficient for pain management, subject can receive a dose of 10 mg PRN. If a subject is unable to tolerate PO medication or fails the PO oxycodone rescue, IV morphine (initiated at 1 to 2 mg) or hydromorphone (initiated at 0.3 to 0.5 mg) may be administered PRN.

All surgical and postsurgical opioid and other analgesics (pain medications) administered must be documented through Day 14 postsurgery. Additionally, an unscheduled pain intensity score using a 10 cm VAS must be completed immediately prior to any rescue medication while in the hospital.

Permitted medications for the prevention and treatment of possible adverse events (AEs) of medications include the following (to be used at the discretion of the study site principal investigator):
Ondansetron 4 mg IV immediately after delivery of the baby
Ondansetron 4 mg IV (should not exceed a maximum of 12 mg in a 24-hour period) for intraoperative and postoperative nausea and vomiting
Metoclopramide 10 mg PO PRN for nausea and vomiting
Nalbuphine IV 2.5 mg PRN pruritus
Naloxone IV 50 to 100 mcg PRN pruritus

1. A method of reducing an amount of a non-opioid analgesic administered to a subject following cesarean section surgery, the method comprising administering into the subject a pharmaceutical composition comprising: a) a multivesicular liposome comprising: at least one amphipathic lipid, and at least one neutral lipid; and b) an aqueous phase comprising bupivacaine phosphate, wherein the aqueous phase is encapsulated within the multivesicular liposome.

2. A method of reducing an amount of a non-opioid analgesic administered to a subject following cesarean section surgery, the method comprising administering into the subject a pharmaceutical composition comprising multivesicular liposomes encapsulating bupivacaine phosphate, said multivesicular liposomes comprising:
bupivacaine or a salt thereof;
phosphoric acid;
a lipid component comprising at least one amphipathic lipid and at least one neutral lipid lacking a hydrophilic head group; and,
optionally, a cholesterol and/or a plant sterol wherein said multivesicular liposomes are made by a process comprising:
a) preparing a first aqueous component comprising phosphoric acid;
b) preparing a lipid component comprising at least one organic solvent, at least one amphipathic lipid, and at least one neutral lipid lacking a hydrophilic head group;
c) mixing said first aqueous component and said lipid component to form a water-in-oil emulsion, wherein at least one component comprises bupivacaine or a salt thereof;
d) mixing said water-in-oil emulsion with a second aqueous component to form solvent spherules; and
e) removing the organic solvent from the solvent spherules to form multivesicular liposomes encapsulating bupivacaine phosphate.

3. The method of claim 1, wherein the aqueous phase further comprises hydrochloric acid.

4. The method of claim 1, wherein the amphipathic lipid is selected from the group consisting of phosphatidylcholines, phosphatidylethanolamines, sphingomyelins, lysophosphatidylcholines, lysophosphatidylethanolamines, phosphatidylglycerols, phosphatidylserines, phosphatidylinositols, phosphatidic acids, cardiolipins, diacyl dimethylammonium propanes, and stearylamines.

5. The method of claim 1, wherein the neutral lipid is at least one triglycerides.

6. The method of claim 1, wherein the pharmaceutical composition comprises a therapeutically effective amount of bupivacaine phosphate.

7. The method of claim 1, wherein the pharmaceutical composition comprises bupivacaine phosphate in an amount equivalent to from about 20 mg to about 300 mg of bupivacaine.

8. The method of claim 7, wherein the pharmaceutical composition comprises bupivacaine phosphate in an amount equivalent to from about 133 mg to about 266 mg of bupivacaine.

9. (canceled)

10. The method of claim 1, wherein the method comprises administering the pharmaceutical composition by transversus abdominis plane (TAP) block.

11. The method of claim 10 wherein the pharmaceutical composition is administering following completion of the cesarean section surgery.

12. (canceled)

13. (canceled)

14. The method of claim 1, wherein the method comprises administering an opioid to the subject following completion of the cesarean section surgery.

15. The method of claim 14, wherein the method comprises less than 200 mg of an opioid to the subject following completion of the cesarean section surgery.

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. The method of claim 1, wherein the subject has an AUC for VAS pain intensity scores over the first 72 hours following completion of the cesarean section surgery of from about 100 to about 200.

32. (canceled)

33. (canceled)

34. The method of claim 1, wherein the subject has a distress from itchiness score as determined by the OBAS scale of less than 4.

35. (canceled)

36. The method of claim 1, wherein the plasma concentration of bupivacaine in the subject after about 120 hours following completion of the cesarean section surgery is about 150 ng/mL to about 250 ng/mL, for an amount of the pharmaceutical composition that is equivalent to about 133 mg of bupivacaine.

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. The method of claim 31, wherein the subject has an AUC for VAS pain intensity scores over the first 72 hours following completion of the cesarean section surgery of from about 125 to 175.

54. The method of claim 31, wherein the subject has an AUC for VAS pain intensity scores over the first 72 hours following completion of the cesarean section surgery of from about 140 to 160.

55. The method of claim 34, wherein the subject has a distress from itchiness score as determined by the OBAS scale of 2.

56. The method of claim 36, wherein the plasma concentration of bupivacaine in the subject after about 120 hours following completion of the cesarean section surgery is about 175 ng/mL to about 225 ng/mL.
